SEQUENCE LISTING (79 sequences)

SEQ ID NO: 1 - protein, 448 aa, Pelotomaculum thermopropionicum
MSHSLREWLAFLEGKGKLKRVRKEVDPVFEIAALGKQADGICSLLFERVKGYAVPVVTGLAGDRELFAAAMSVPVEGMLEKLAAAVENPVPCRLVSPDGAPVKECIIRENIDLLKMLPIPTHHAGDAGPYITAAILIARDPDSGVRNVSIHRLQVTGPDRLGILILPRHLWHFFGKAERAGRPLEIALAIGVHPAVLLASQATTRLGVDELEIASALLPQPLELVKCETVDVEVPAGAEIVIEGKILPGVREVEGPFGEYPRYYGPAAPRPVVEVTAVTHRRQPVYHTIIPASREHLLLGGIAREAVLLQTVRQAVPTVKNVHLTPGGSCRYHAVISIEKKHEGEAKNAIFAAFTSSSEVKHVVVVDHEINIFDPEEVEWAVATRCQAGRDVFIVKDAMGNRLDPSSRDGVSDKMGIDATIPLNLPGERFERISIPGLDKIKLADYLE

SEQ ID NO: 2 - protein, 468 aa, Geobacillus kaustophilus
MRAKTFRTWLEYMQSTERLAVIDKRVKLKYEVASIAKKLDGKKAVYFTQVEDYSIPVVSGICTTRQQFAEALETDQSGLIPKFIQAVSNPLPYRLVDKKDAPVKENVILKNIDLMEILPIPIHHEKDSGHYITAGLLIVRDPLTRKQNVSIHRLQVSSKDRLGILILPRHTFHLYRQAEEAGKPLECAIAIGVDPITLLASQASTPFGIDELEIASALRGEPLEVVRCETVDIDVPAHAEIVLEGKILPRVREPEGPFGEFPKYYGSRSDKEVVQITGITHRNHPIFHTIVPAGYEHLLLGGIPREASLFQSVKQTVPSVKSVHMSLGGTCRYHAIISIKKRNEGEAKNAILAAFANSFDIKHVVVVDEEIDIFNMEEVEWAIATRFQADRDLIVIHGAQGSKLDPSTNNGVGCKMGLDCTVPLDSEPMKYLRIEIPGYNDLDVDEYINQESTGLKPEHLEKNNAASI

SEQ ID NO: 3 - DNA, 1347 bp, Pelotomaculum thermopropionicum
atgtcccactccctgcgtgaatggctggcgttcctggaaggcaaaggcaaactgaaacgtgtgcgtaaagaagtcgatccggtgttcgaaattgcggccctgggcaaacaggcggatggtatctgctccctgctgtttgaacgtgtgaaaggctatgcagttccggtggttacgggtctggcaggtgatcgtgaactgttcgcagctgcgatgtctgtcccggtggaaggcatgctggaaaaactggccgcagctgtggaaaacccggttccgtgccgtctggtgtcaccggatggtgcgccggttaaagaatgtatcatccgcgaaaacatcgacctgctgaaaatgctgccgatcccgacgcatcacgcaggcgatgctggtccgtacattaccgcggccattctgatcgcccgtgatccggactcgggtgttcgtaacgtcagcatccatcgtctgcaggtgaccggtccggatcgtctgggtattctgatcctgccgcgccatctgtggcactttttcggtaaagcagaacgtgcaggtcgtccgctggaaattgcactggctatcggtgtccatccggcagtgctgctggcatcccaggctaccacgcgtctgggtgtggacgaactggaaattgcgtcagccctgctgccgcaaccgctggaactggtgaaatgtgaaacggttgatgtcgaagtgccggcaggcgctgaaattgttatcgaaggcaaaatcctgccgggtgtgcgcgaagttgaaggcccgtttggtgaatatccgcgttattacggtccggcagctccgcgtccggtcgtggaagtcaccgcagtgacgcatcgtcgccagccggtgtaccacaccattatcccggccagccgtgaacatctgctgctgggcggtattgcgcgtgaagccgtcctgctgcagaccgttcgccaagcagttccgacggtcaaaaatgtgcacctgaccccgggcggttcttgccgttatcatgcggtgattagtatcgagaaaaaacacgaaggcgaagcaaaaaacgctatttttgcggccttcaccagcagcagcgaagtgaaacatgttgtcgtggttgatcacgaaattaatatctttgacccggaagaagttgaatgggcagtcgcaacccgttgtcaagcaggtcgcgatgttttcatcgtcaaagacgctatgggtaaccgtctggacccgtcctcacgcgatggcgtgtcggacaaaatgggtattgatgccaccatcccgctgaatctgccgggcgaacgtttcgaacgcattagcatcccgggtctggataaaattaaactggcagactacctggaataa

SEQ ID NO: 4 - DNA, 1407 bp, Geobacillus kaustophilus
atgagggcaaaaacatttcgtacatggttagaatatatgcaatcaactgaacgtctagcggttatcgataaaagggttaaactaaagtatgaagtagcttccattgcgaaaaaattagacggaaagaaagcagtgtattttacacaagtagaggattattcgatcccagttgtatctggaatttgtaccacacgacagcaatttgcagaggctttagaaacagatcagtctgggctgatacctaaatttatccaagctgtttctaatcctcttccttatcgtcttgtagataaaaaagatgctcctgtgaaagaaaatgttattttgaaaaatatcgatctcatggagattttacctataccgattcatcatgaaaaggattcaggtcattatattacggcgggattactaatcgttcgggatccattaacacgaaaacagaacgtttctatccatcgtttgcaagtatccagtaaagacagacttgggattcttatcctcccgcgtcatacatttcacttatatagacaggcggaagaagcaggaaaaccattagaatgtgcaatagccataggagttgaccctattaccctactagcatcacaggcaagcaccccctttgggatagatgaattagagatcgctagtgccttacgtggcgaaccgcttgaagtcgtacgatgtgagacagtagacattgacgttccagctcacgcggaaatcgtactagagggaaagattcttcctcgagttcgtgaaccagaaggtccttttggggaatttccgaaatattatggatctcgtagtgacaaggaggtagtacaaattacaggaattacccaccgaaaccatcctatttttcatacgatcgtacctgctggatatgaacatttgttacttggcggtatccctcgagaagccagtctttttcaaagtgttaaacaaacggtgccatcagtgaaatcggttcatatgagtcttggaggcacttgccgttaccatgcaatcatctctattaagaagcgaaatgaaggagaggccaaaaacgcaatattggctgcattcgccaatagttttgatatcaaacatgtagttgttgtagacgaggaaattgatattttcaacatggaagaggttgaatgggctattgctacacgtttccaagcggatcgggatttaatcgtgattcatggtgcccaaggctcgaaacttgatccgtcaactaataatggggttggttgtaaaatggggctagactgtactgttccattggatagtgaacccatgaaatatttacgtatagagatcccaggttacaacgatttggatgtagatgaatatatcaatcaagaatcaactggtttgaagccagaacatcttgagaaaaacaatgccgcctctatatag

SEQ ID NO: 5 - protein, 448 aa, Desulfotomaculum geothermicum
MAYSLRDWLAFLDREGKLKRIKKEVDPVFEIAALGKQADGIYSLLFERVKGYEMPVVTGLVGERGLFAVAMSTTVEGMLDKFAAAVENPVPCRLVSSNSAPVKECIVNKDIDLFKMFPIPTHHAKDAGPYITAAILIAKDPDTGVRNISIHRLQISGPNKLGILILPRHLWHFFSKAEQKGRPLEIALAIGVHPLVLLASQATTRIGVDELEIASALLPQPLELVKCETVDLEVPAGAEIVIEGRLLPGVREVEGPFGEYPKYYGPASPRPVVEVMAVTHRRQPIYHTIIPATREHVLVGGIAREAVLLQIVRQAVPTVKNVHLTLGGSCRYHAVISIEKKHEGEAKNAIFAALASCSEVKHVVVVDHDVNIFDPEDVEWAIATRCQAGRDVFIVTGAMGNKLDPSSNDGISDKMGIDATIPLELPAERFEKISIPEYNDIKLDDYLD

SEQ ID NO: 6 - protein, 447 aa, Desulfotomaculum nigrificans
MICTSLRQWLSLLEEKGLLKQVNKLVDLKYELAALGKKADGKYALRFNNVGDYKIPVVTEIAGGREFFAMAMDVPVDQVAEHFARAQANPIDCTVVDASKAPVKEVVSQNVDLSTLPIPIHHEKDSGPYITAGVLVAKDPKTGIRNISIHRLQVLGPNRLGILILPRHLFHFFREAESMGKPLEVAIAIGLDPLVLLASQALTPPGVDELTIASALYGKPLELVKCETVDLEVPAQAEIVLEGKLLPGIREMEGPFGEYPKYYGPASPKPVIELTAMTSRKDPIYQTIVPATREHLLLGAIPREGGLFQIVKNTVPTVCAVHLTPGGTCRYHVVISIDKQNEGEAKNAIFAALSSSQEIKHVVVVDKEVNIFDPEDVEWAIATRCQADRDVFVVAGAMGNKLDPSSDDGLSAKMGIDATVPLNAEPGRFERIRIPGEDTISLEDYIE

SEQ ID NO: 7 - protein, 447 aa, Desulfotomaculum nigrificans CO-1-SRB
MICTSLRQWLSLLEEKGLLKQVNKLVDLKYELAALGKKADGKYALRFNNVGDYKIPVVTEIAGGREFFAMAMDVPVDQVAEHFARAQANPIDCTVVDASKAPVKEVVSQNVDLSTLPIPIHHEKDSGPYITAGVLVAKDPKTGIRNISIHRLQVLGPNRLGILILPRHLFHFFREAESMGKPLEVAIAIGLDPLVLLASQALTPPGVDELTIASALYGKPLELVKCETVDLEVPAQAEIVLEGKLLPGIREMEGPFGEYPKYYGPASPKPVIELTAMTSRKDLIYQTIVPATREHLLLGAIPREGGLFQIVKNTVPTVCAVHLTPGGTCRYHVVISIDKQNEGEAKNAIFAALSSSQEIKHVVVVDKEVNIFDPEDVEWAIATRCQADRDVFVVAGAMGNKLDPSSDDGLSAKMGIDATVPLNAEPGRFERIRIPGEDTISLEDYIE

SEQ ID NO: 8 - protein, 460 aa, Desulfotomaculum thermosubterraneum
MITVSYHTLRSWLEALETRGYLKKIKREVDPVFELAAVAKKADGQWAVKFEKVKGYSMPVVAGILSTRQMIAEAMGVSIAEVVTKFAAAQANPVPCRLISREQAPVKEVIIRDHIDIVSMFPVPTHHEKDAGPYITAALLIAKDPSTGVRNVSIHRLQVLGPNKLGILILPRHLWHIFQKAEENDRPLEVALVIGVDPLTLLASQAITPFGVDELEIAGALHGEPLQLVKCETVDIEVPAHAEIVLEGRLLPGVREMEGPFGEYPKYYGPASKKPVIVIDAVTCRRQPIYQTIVAATKEHLLLGAIPREAGLFQIVKQAVPTVQAVRLTPGGACRYHAVIAIDKRNEGEAKNAMFAAFASSAEIKHVVVVDKDVDIFNSEDVEWAIATRCQAGRDVIIISGAHGNKLDPSSDDGVSDKMGIDATVPLGAPAERFEKIKIPDFEKIKLEDYFEDHEQKTVL

SEQ ID NO: 9 - protein, 457 aa, Desulfotomaculum thermosubterraneum DSM 16057
MSYHTLRSWLEALETRGYLKKIKREVDPVFELAAVAKKADGQWAVKFEKVKGYSMPVVAGILSTRQMIAEAMGVSIAEVVTKFAAAQANPVPCRLISREQAPVKEVIIRDHIDIVSMFPVPTHHEKDAGPYITAALLIAKDPSTGVRNVSIHRLQVLGPNKLGILILPRHLWHIFQKAEENDRPLEVALVIGVDPLTLLASQAITPFGVDELEIAGALHGEPLQLVKCETVDIEVPAHAEIVLEGRLLPGVREMEGPFGEYPKYYGPASKKPVIVIDAVTCRRQPIYQTIVAATKEHLLLGAIPREAGLFQIVKQAVPTVQAVRLTPGGACRYHAVIAIDKRNEGEAKNAMFAAFASSAEIKHVVVVDKDVDIFNSEDVEWAIATRCQAGRDVIIISGAHGNKLDPSSDDGVSDKMGIDATVPLGAPAERFEKIKIPDFEKIKLEDYFEDHEQKTVL

SEQ ID NO: 10 - protein, 457 aa, Desulfotomaculum kuznetsovii DSM 6115
MSYHTLRSWLEALETRGYLKIVKREVDPFFELAAVAKKADGQWAVKFEKVKGYSMPVVAGILSTRQMIAEAMGVSIAEVVTKFAGAQANPVPCRLISREQAPVKEVIIRDHIDIVSMFPVPTHHEKDAGPYITAALLIAKDPSTGVRNVSIHRLQVLGPNKLGILILPRHLWHIYQKAEENDRPLEVALVIGVDPLTLLASQAITPFGFDELEIAGALHGEPLQLVKCETVDIEVPAHAEIVLEGRLLPGVREMEGPFGEYPKYYGPASKKPVIVIDAVTCRRQPIYQTIVAATKEHLLLGAIPREAGLFQIVKQAVPTVQAVHLTPGGACRYHAVIAIDKRNEGEAKNAMFAAFASSAEIKHVVVVDKDVDIFNSEDVEWAIATRCQAGRDVIIISGAHGNKLDPSSDDGISDKMGIDATVPLGAPAERFEKIKIPDFEKIRLEDYLEDHEQKTVL

SEQ ID NO: 11 - protein, 450 aa, Calderihabitans maritimus
MSFRSLRSWLKTLETKGYLKKVKREVDPVYELAAVAKKADGQWAVKFEKVKGYSVPVVAGILHNRQLIAEAMKINVAELVTKFAEAQANPLPCCLIDREKAPVKEVIVRDNIDLGSMFPIPTHHEKDAGPYITAALLIAKDPCTGVRNVSIHRLQLLGPNRLGILILPRHLWHIFRQVEEKDQPLEIALVIGVDPLTLLASQAITPFGVDELEIASALHGEPLQLVKCETVDIEVPAHAEIVLEGRLLPGVREIEGPFGEYPKYYGPASKKPVIVIDAVTCRRRPIYQTIVAATREHLLLGAIPREATLFQMVRQAVPTVRAVHLTPGGTCRYHAVIAIDKRHEGEAKNAIFAAFASSAEVKHVVVVDKDVDIFNPEDVEWAIATRCQASKDVIIISQAHGNRLDPSSDDGLSDKMGIDATVPLSAPAERFERIRIPGFEKIRLEEYLGD

SEQ ID NO: 12 - protein, 447 aa, Moorella mulderi
MVANSLRQWLAELDRKGLLKQVNKEVDLHYELAAVGKKADGRYALQFNKVRNSSIPVVTGIAGTREIMAAAMGVSIKEVAERFAQAQANPVECVLVASGKSPVKENVNYEVDLGSLPIPVHHEKDGGPYITAGVLVAKDPRTGIRNISIHRLQVLGPNRLGILILPRHLSHFFRAAEEAGRPLEVAIAIGLDPLLLLASQALTPPGFDEFTIAGALYGQPLELVKCETVDLEVPAQAEIVLEGRLLPGVREIEGPFGEYPKYYGPASPKPVIELTAITSRRNPIYQTIVPATMEHLLLGAIPREGGLLQVIKNAVPNVKGVHLTPGGTCRYHAVIAIDKQNEGEAKNAIFAAFSSSQEIKHVVVVDKDVDIFNPEDVEWAIATRCQAGRDVFIVERALGNKLDPSSDNGLSDKMGIDATVPLDAEPGRFERIRIPGEEAIRLEDYLD

SEQ ID NO: 13 - protein, 451 aa, Desulfurispora thermophila
MSYRTLRSWLEALETRGYMKKVKREVDPVFELAAVAKKADGQWAVKFEKVKGYSMPVVAGILSTRQMIAEAMGVSITEVVPIFAGAQANPVPCRLIDRAQAPVKEVVIREHFDLVSMLPIPTHHEKDAGPYITAALLIAKDPSTGVRNVSIHRLQVLGPNKLGILILPRHLWHIFQKTEENDQPLEVALVIGVDPLTLLASQAITPPGVDELEVAGALHGESLQLVKCETVDIEVPAHAEIVLEGRLLPGVREMEGPFGEYPKYYGPASKKPVIVIEAVTCRRQPIYQTIVAATKEHLLLGAIPREAGLFQIVKQAVPTVQAVHLTPGGACRYHAVVAIDKRNEGEAKNAMFAAFASSAEIKHVVVVDKDVDIFNSEDVEWAIATRCQASRDVIMISGAHGNKLDPSSDDGVSDKMGIDATVPLGAPAERFEKIKIPDFEKIRLEDYLEER

SEQ ID NO: 14 - protein, 450 aa, Moorella thermoacetica
MVCTSLRQWLAELEKKELLRHVHKEVDLRYELAAVGQKADGCYALQFHKVRNASMPVVTGIAATRDILALAMGVPREEIVEHFARAQANPVKCMLVDAKHAPVKEVISHKVDLGTLPIPVHHEKDGGPYITAGVLVAKDPQTGTRNVSIHRLQVLGPNRLGILILPRHLHHFFQAAEASNNPLEVAIAIGLDPIVLLASQALTPPGFDEFTIAGALYGQPLELVKCETVDLEVPAQAEIVLEGRLLPGVREVEGPFGEYPKYYGPASPKPVIELTAMTSRRDPIYQTIVPATREHLLLGAIPREGGLLQIIRNTVPNVKAIHLTPGGTCRYHVVISIDKQNEGEAKNAIFAAFSSSQEIKHVVVVDKDVDIFDPEDVEWAIATRCQAGRDVFIVERALGNKLDPSSDNGLSDKMGIDATAPLQAEPGRFEKIRIPNADSIRLEDYIETKS

SEQ ID NO: 15 - protein, 447 aa, Moorella glycerini
MALTSLRQWLAELQQRGFLKHVNKEVDLHYELAAVGKKADGQFALQFNNAGNSSIPVVTGIAGTREILALAMGVARDQVAERFAQAQANPVECVLVASGKAPVKENVNYDVDLGSLPIPVHHEKDAGPYITAGVLVAKDPRTGIRNISIHRLQVLGPNRLGILILPRHLSHFFRAAEEAGRPLEVAIAIGLDPLLLLASQALTPPGFDEFTIAGALYGQPLELVKCETVDLEVPAQAEIVLEGMLLPRVREMEGPFGEYPKYYGPASPKPVIELTAMTSRRNPIYQTIVPATMEHLLLGAIPREGGLLQVIKNAVPNVKGVHLTPGGTCRYHAVITIDKQNEGEAKNAIFAAFSSSQEIKHVVVVDKDVDIFNPEDVEWAIATRCQAGRDVFIVERALGNKLDPSSDNGLSDKMGIDATVPLDAEPGRFERIRIPGEEAIRLEDYLD

SEQ ID NO: 16 - protein, 447 aa, Peptococcaceae bacterium BRH_c23
MRARSLRQWLQVLNTDGMLKHVDKKVNLSYELAAVGKKADGRFAVQFDKPGDSQVPVVTGIGGSRELLARAMGVSVDRVAEHFAWAQANPMECTIVEAGAAPVKERVTHDVDLGTLPIPVHHEKDGGPYITAGVLIAKDPKTGVSNLSIHRLHVLGPNRLGILILPRHLSHFHRMAEAEGKPLEVAIAIGLDPILLLASQALTSPGYDEYTIASALYGQPLELVKGETVDLEVPAQAEIVLEGYLLPAVREMEGPFGEYPKYYGPASPKPVIELTAMTSRMDPIFQTIVPATKEHLLLGSVPREGGLIQIIRNAVPNTRSVHLTPGGTCRYHLAISIDKQNEGEAKNAMFAAFGSSQEIKHVVVVDKDVDIFNPEDVEWAVATRCQAGRDVFIVERALGNKLDPSSDDGISDKMGIDATVPSDAPVGRFERIRIPGEDKIKLEDYCS

SEQ ID NO: 17 - protein, 467 aa, Fictibacillus enclensis
MKARTFRTWLDHLQSKGRLAVINKKVSPDFEIAAVAKKLDGQKAAYFSNVDGYKVPLVSGICSSRQDFADALETDEKGIIPKFSKAVSIPKPCRLIEKEDAPVKENIITENIDILKTFPIPIHHEMDSGHYISAALFITRDPDTRKQNVSIHRLQISGKDKLGVLLLPRHTHHLYKQAEEQGKPLECAIVIGVDPLTLLASQASTPFGVDELEIASALRSEPLETVRCETVDIDVPARAEIVIEGKILPHIREPEGPFGEFPKYYGPRSDKEIVQVTAISHRDNPIFYTIVPAGYEHLLLGGIPREASLLESIRQTVPSVAAVHMSPGGTCRYHAIVSIKKRNEGEAKNAILAAFANSFDIKHVVVADEDVDIFNMEEVEWAIATRFQASKDMVVIHDAQGSKLDPSTDNGLGSKTGFDCTIPLNSEPMRYKRITIPGYDELNLQDYLSHDASESKKESFETDKVLT

SEQ ID NO: 18 - protein, 450 aa, Desulfotomaculum putei
MVTSSLRDWLAYLENEGKIKTVSKEISLKFEMPAVIKKLDGKAAVKFKKPKGYNIPVVSGIAYDRDLFAKALGTTKNNVARKISECQKQPIPCQVVTREEAPVMKNINIDDVNLMTLPIPIHHEKDAGHYITAGLLICKDPETGKRNVSIHRLQVFSKNEIGILILPRHLSQLYMKAEKKNEPLDIAIAIGVDPVLLLASQAIVPLGVDELEIANAIKNGGQKLVKCQTVDVEVPAEAEIVLEGKLLPKVRKVEGPFGEFPKYYGPASEKPVIKITAICHRDEPIYHTILPATKEHLLLGGLAREATLYELVKQTVPTVKDVHLTIGGTCRYHAVISIDKKHEGEAKNAMFAAFASMQEVKHVVVVDSDVDIFDIEEVEWAIATRCQMAKDIMLVSGSLGSKLDPSTNDGISDKMGIDATCPLNAPPEKYETIKIPGYEELNIEDYLDNE

SEQ ID NO: 19 - protein, 449 aa, Oscillibacter sp. PC13
MKPILREWLAKMEQDGMMKRISREVDPINELAAVGKKLEPQYGALFEHVRGSSMPVVTGVITSRDGMAKSMGMSYEELLQDFNHALSNPTPCKVLEKEQGPFAIKENILTGDQVDLGILPACLHHEKDSSRYLTAALCIAKDPETGIRNVCIHRHEIKDRNHLGALLLPRHTNQMFSKAEAMGKPLEIALAIGVHPSLLLASQATTALGVDEFEIAGSLLGEPVELVKCETVDLEVPVESEIIIEGKILPNVREEEGPFGEYPKTYGAKAPRHVIEITAITYRNNPIYHTIIPATMEHLLLGGISREATMMQVIRQATPNVKKVHITPASGCRYHVVIQLDQKHEGEAKNAMFAAFTSSTEVKHVVVVDTDIDIYDMQDVEWAISNRVQAARDVFIIPNAMGNKLDPSSRGGASDKMGIDATIPMSDKRERFQKIHIAGYEDINLKDYL

SEQ ID NO: 20 - protein, 445 aa, Desulfotomaculum putei DSM 12395
MRDWLAYLENEGKIKTVSKEISLKFEMPAVIKKLDGKAAVKFKKPKGYNIPVVSGIAYDRDLFAKALGTTKNNVARKISECQKQPIPCQVVTREEAPVMKNINIDDVNLMTLPIPIHHEKDAGHYITAGLLICKDPETGKRNVSIHRLQVFSKNEIGILILPRHLSQLYMKAEKKNEPLDIAIAIGVDPVLLLASQAIVPLGVDELEIANAIKNGGQKLVKCQTVDVEVPAEAEIVLEGKLLPKVRKVEGPFGEFPKYYGPASEKPVIKITAICHRDEPIYHTILPATKEHLLLGGLAREATLYELVKQTVPTVKDVHLTIGGTCRYHAVISIDKKHEGEAKNAMFAAFASMQEVKHVVVVDSDVDIFDIEEVEWAIATRCQMAKDIMLVSGSLGSKLDPSTNDGISDKMGIDATCPLNAPPEKYETIKIPGYEELNIEDYLDNE

SEQ ID NO: 21 - protein, 447 aa, Sporomusa acidovorans
MICTSLRDWLTLLEQEKILKTVTKQVDLKHELAALGKKADGKYALKFVNTGGSMPVVTGFGGSRALLAKAMGVEVHQVAEHFAQAQENPMKCVLVDKANAPVKEFVTFDVDLAKLPIPIHHEKDSGAYITAALLIAKDPETGARNVSIHRLQVSGANRLGILILPRHLHNFYQKAEAENKPLEIALAIGVDPILLLASQALAPLGFDEFTIASRLYGKPLELVKCETVDLEVPAQAEIVLEGRLLPNVREMEGPFGEYPKYYGPASLKSVIELTAMTSRKNPIYHTIVPATMEHCLLGAIPREGGMLQLIRNTVPTTLGVHLTAGGTCRYHAVVKIDKKSEGEAKNAIFAALSSSHEIKHVVVVDKDVDIFNMEDVEWAIATRSQAGRDVFIVERAAGNKLDPSTDDGVCDKMGIDATVPLDAPAGRFERIRIPGEDQLDLTEYILD

SEQ ID NO: 22 - protein, 467 aa, Fictibacillus solisalsi
MKARTFRTWLDHLQSKGRLAVINKKVSPDFEIAAVAKKLDGQKAAYFSNVDGYKVPLVSGICSSRQDFADALETDEKGIIPKFSKAVSIPKPCRLLEKEDAPVKENIITENIDILKTFPIPIHHEMDSGHYITAALFITRDPETRKQNVSIHRLQISGKDRLGALLLPRHTHHLYKQAEEQGKSLECAIVIGVDPLTLLASQASTPFGVDELEIASALHSEPLETVRCETVDIDVPARAEIVIEGKILPHIREPEGPFGEFPKYYGPRSDKEIVQVTAISHRHHPIFYTIIPAGYEHLLLGGIPREVSLLESIRQTVPSVAAVHMSPGGTCRYHAIVSIKKRNEGEAKNAILAAFANSFDIKHVVVADEDVDIFNMEEVEWAIATRFQASKDMVVIHDAQGSKLDPSTDNGLGSKTGFDCTIPLNSEPMRYKRITIPGYDELNLQDYLSDDASESKRESFETDKVLT

SEQ ID NO: 23 - protein, 447 aa, Desulfovibrio dechloracetivorans
MQPILRTWLRQLEEEGLLKRIKKAVDPAFELACVGKKLEPDYGVLFENVNGFSMPVVTGLATSRDSMAKALGLTYTELTERFSEAITSPTPCTVVEPEGLAVKERKFIGDDVDLRSLPACVHHAKDSGPFLTAAMCIVRDPETGIRNVSIHRHEVKDRNHLGALLLPRHTSQIFNKLESQGKPLEVALVIGAHPALLLSSQATTRLGVDELEIAGTLLGEPLKVTPCETVDLEVPVESEIVIEGRLLANTREDEGPFGEYPRTYGPRGKRHVIEVTAITHRAEPIYHTIIPASMEHLLLGGVSREASMLQLIRQATPNIIDVRLTPAGGCRYHAVIRLDQKHEGEAKNAIFAALASSTEVKHVVAVNPDIDIDDMRDVEWAIANRVQGGRDVFIVPGAMGNKLDPSSRDGVSDKIGIDATIPLGQDCERFEKMCIPGYESLCLEDYI

SEQ ID NO: 24 - protein, 464 aa, Thermoflavimicrobium dichotomicum
MKAKTFRTWLEHLQSVGRLAVIDKKVSLEFELAAIAKKLDGKKSTYFTQVEDYAVPVVSGICSTRVDFAEALETDQYEMIPKFIKAVTSPLPCRLVEDEEAPVKENIIKDNIDLMKMFPIPVHHEKDSGNYITAGLFIVRDPVTRKQNVSIHRLQVSGKDKLGALILPRHTFHLYKQAEEAGRPLECAIAIGVDPVTLLASQASTPFGVDELEIAGALRNEPLEVVRCETVDIDVPAYAEIVLEGKILPHVREPEGPFGEFPKYYGPRSDKEVVQITAVTHRNNPIFYTIVPAGYEHLLLGGIPREASLFQTIRQTVPTVKAVHMSPGGTCRYHAIVSIKKRNEGEAKNAIFAAFANSFDIKHVVVVDEEVDIFNMEEVEWAIATRFQAEKDLIVVHGAQGSKLDPSTNNGVGSKMGFDCTVPLNSEPMKYLRIQIPGYDQINLEEYINSEASIQSVHLEGEKR

SEQ ID NO: 25 - protein, 449 aa, Oscillibacter sp. 1-3
MKPILREWLTEAEARGLMKRVRREVSPIHELAAVGKLLEPQYGAFFERVAGSAIPVVTGLAASREAMAQSIGLSVRELPDRFNDALTRLTPCKTLNREDGPFAVKAHILTGEQVDLGILPACLHHEKDSARYLTAALCIVKDPETGIRNVSIHRHEIKDRSHLGALLLPRHTNQIFRRAEALGRPLEIALAIGVHPALLLSSQATTRLGVDEFEIGGTLLGEPIELVPCETVDLEVPVECEIVIEGRILPNVRENEGPFGEYPRTYGPRAPRHVIEVTAITHRDTPIYHTIIPASMEHLLLGGIPREAAMLQLVRQTTPNVKSVHITPAGGCRYHAVIQLDQQYKGEAKNAIFAAFASSSEIKHVTVVDTDIDIFDTQDVEWALANRVQAGRDVFVVTGAMGNKLDPSSADGVSDKLGIDATIPVDAERGRFQKIHIAGMENIRLEDYL

SEQ ID NO: 26 - protein, 465 aa, Bacillus sp. FJAT-20673
MKAKTFRTWIEHLQATHRLAVIDRKVSLQYEIAKIAKKLDGKKATYFTSVEDYQVPVVSGICSTREDFAEALETDQYGVISKFTEAVESPVPCRALNKQQAPIKENIILDNIDLMKMLPIPVHHEKDSGNYITAGLFIVRDPVTRKQNVSIHRLQISGGNKIGALILPRHTFHLYKQAEEAGRPLECAIVIGVDPVTLLASQASTPFGVDELEISSALRGEPLEVVRCETVDIDVPAYAEIVLEGKILPHVREPEGPFGEFPKYYGPRSDKEVIEITAVTHRNNPIFYTIVPAGYEHLLLGGIPREASLFQSIRQTVPSVKAVHMSPGGTCRYHAIVSINKRNEGEAKNAILAALANSFDIKHVVVTDEEVDIFNMEEVEWAIATRFQADRDLVVVHEAQSSKLDPSTRDGVGSKMGFDCTIPLDSEEMQYLRVQIPGYNECNLDDYINPDKLIQSVHLEGDIDF

SEQ ID NO: 27 - protein, 465 aa, Bacillus simplex
MKAKTFRTWIEHLQATHRLAVIDRKVSLQYEIAKIAKKLDGKKATYFTSVEDYQVPVVSGICSTREDFAEALETDQYGVISKFTEAVASPVPCRALNKQQAPIKENIILDNIDLMKMLPIPVHHEKDSGNYITAGLFIVRDPVTRKQNVSIHRLQISGGNKIGALILPRHTFHLYKQAEEAGRPLECAIVIGVDPVTLLASQASTPFGVDELEISSALRGEPLEVVRCETVDIDVPAYAEIVLEGKILPHVREPEGPFGEFPKYYGPRSDKEVIEITAVTHRNNPIFYTIVPAGYEHLLLGGIPREASLFQSIRQTVPSVKAVHMSPGGTCRYHAIVSINKRNEGEAKNAILAALANSFDIKHVVVTDEEVDIFNMEEVEWAIATRFQADRDLVVVHEAQSSKLDPSTRDGVGSKMGFDCTIPLDSEEMQYLRVQIPGYNECNLDDYINPDKLIQSVHLEGDIDF

SEQ ID NO: 28 - protein, 449 aa, Dethiosulfatibacter aminovorans
MDLNLRNWIEVMKKEKVLKTVSKEVSTEFEIAAIGKKLEPDYGVMFNNVKGYDVPVITGLAGTREKMASSLSLTVEELMERFNNALSLPTPCSIVPSEGLGIKENIYIGDDVDIERILPACVHHEKDSGKYITSGMLIVKDPETGIRNVAIHRHEIKDKNHLGALLLPRHTHHIFDRAEKTGKPLEVALIIGTHPVLLLASQATTRLGIDEFEIAGSLIGKPVAMTKCETVDLEVPIECEYVLEGKILPNIRKDEGPFGEYPKTYGPKRPRHVIEITAITHRDNPIYHTIIPATMEHLLLGAIPREATMFQIVKQAVPSAFGVHLTPAGGCRYHVIIGIDKKNEGEAKNAIFAAFASSSEVKQVVVVDKDIDIFDSKDVEWAIANRVQANNDVIIVKSAMGNKLDPSSDEGVSDKMGIDATVPLNADPERFQKINIPMYDEIILEDYLD

SEQ ID NO: 29 - protein, 468 aa, Aeribacillus pallidus
MRAKTFRTWLEYLQAQDRLAVIDRPVSLRFELAALAKKLDGRKAVFFSQVDDYQVPVVSGICSTREQFAEALETDPYGLIPKFAQAVASPVPCRQVKKEDAPVKDHIRLDDIDLMRMLPIPVHHEKDAGSYITAGLFIVRDPVTRKQNVSIHRLQVSGKNRLGVLLLPRHTFHLYKQAEEAGRPLECAIAIGVDPATLLASQASTPFGVDELEIAGALRGEPLEVVRCETVDIDVPAYAEIVLEGRILPNVREPEGPFGEFPKYYGPRSDKEVVEITAVTHRSHPIFYTIVPAGYEHLLLGGIPREASLFQTIRQAVPSVKAVHMSPGGTCRYHAVVSIQKRNEGEGKNAIVAALANSFDIKHVVVVDEDVDIFNMEEVEWAVATRFQAERDLVVVHGAQSSRLDPSTKDGVGSKMGLDCTVPLGSEPMRYLRINIPGYEEIRLEDYLRPGGGTLGETYLERGKKAVR

SEQ ID NO: 30 - protein, 468 aa, Brevibacillus sp. WF146
MRAKTFRTWLEYLQAQDRLAVIDRPVSLRFELAALAKKLDGRKAVFFSQVDDYQVPVVSGICSTREQFAEALETDPYGLIPKFAQAVASPVPCRQVKKEEAPVKDHIRLDDIDLMRMLPIPVHHEKDAGSYITAGLFIVRDPVTRKQNVSIHRLQVSGKNRLGVLLLPRHTFHLYKQAEEAGRPLECAIAIGVDPATLLASQASTPFGVDELEIAGALRGEPLEVVRCETVDIDVPAYAEIVLEGRILPNVREPEGPFGEFPKYYGPRSDKEVVEITAVTHRSHPIFYTIVPAGYEHLLLGGIPREASLFQTIRQAVPSVKAVHMSPGGTCRYHAVVSIQKRNEGEGKNAIVAALANSFDIKHVVVVDEDVDIFNMEEVEWAVATRFQAERDLVVVHGAQSSRLDPSTKDGVGSKMGLDCTVPLGSEPMRYLRINIPGYEEIRLEDYLRPGGGTLGETYLERGKKAVR

SEQ ID NO: 31 - protein, 468 aa, Brevibacillus thermoruber
MRAKTFRTWLEYLQAQDRLAVIDRPVSLRFELAALAKKLDGRKAVFFSQVDDYQVPVVSGICSTREQFAEALETDPYGLIPKFAQAVASPVPCRQVKKEDAPVKDHIRLDDIDLMRMLPIPVHHEKDAGSYITAGLFIVRDPVTRKQNVSIHRLQVSGKNRLGVLLLPRHTFHLYKQAEEAGRPLECAIAIGVDPATLLASQASTPFGVDELEIAGALRGEPLEVVRCETVDIDVPAYAEIVLEGRILPHVREPEGPFGEFPKYYGPRSDKEVVEITAVTHRSHPIFYTIVPAGYEHLLLGGIPREASLFQTIRQAVPSVKAVHMSPGGTCRYHAVVSIQKRNEGEGKNAIVAALANSFDIKHVVVVDEDVDIFNMEEVEWAVATRFQAERDLVVVHGAQSSRLDPSTKDGVGSKMGLDCTVPLGSEPMRYLRINIPGYEEIRLEDYLRPGGGTLGETYLERGKKAVR

SEQ ID NO: 32 - protein, 474 aa, Rhodoplanes sp. Z2-YC6860
MPPPEATRSNTPQPVRTLRDWLDHLAARDRLAVLKPNTRLKFEVAAYSKRLDGLRATVFPKPDGHPIPVVSGLVSDRGWMAEAMGVEPSEVLAHFQNAAENPVPWQEVASGPAQEVVHRQDRGALDLAKILPLPTHNEHDGGPYIAAGIMIVRNPKTGKQNVSIHRCQLTGPNRLGVLVLPRHTFTFQRMAEEAGQPLDAAIVVGVDPLTLLASQAIVPLDHDELEIAGALHGRPLPVVKCVASDIRVPADAEIVIEGRFLPHVREPEGPFGEFPQYYGERAPREVMEIVAVTHRKDAIFHTIVGGGLEHQLLGAIPKEATLLTHLRRNFSNVLDVHLSPGGVMRFHLFVKIRKTQEGQGKNVILGAFAGYFDLKHVIVVDEDVDIHNPTEVEWAVATRFQADRDLVIVPESQGSKLDPSNRDGVGAKMGLDATKPFKAPEMKFKRIYVPGEEAINVADTLKQGGADWRSALKS

SEQ ID NO: 33 - protein, 479 aa, Rhodoplanes sp. Z2-YC6860
MSKHDMPPPEATRSNTPQPVRTLRDWLDHLAARDRLAVLKPNTRLKFEVAAYSKRLDGLRATVFPKPDGHPIPVVSGLVSDRGWMAEAMGVEPSEVLAHFQNAAENPVPWQEVASGPAQEVVHRQDRGALDLAKILPLPTHNEHDGGPYIAAGIMIVRNPKTGKQNVSIHRCQLTGPNRLGVLVLPRHTFTFQRMAEEAGQPLDAAIVVGVDPLTLLASQAIVPLDHDELEIAGALHGRPLPVVKCVASDIRVPADAEIVIEGRFLPHVREPEGPFGEFPQYYGERAPREVMEIVAVTHRKDAIFHTIVGGGLEHQLLGAIPKEATLLTHLRRNFSNVLDVHLSPGGVMRFHLFVKIRKTQEGQGKNVILGAFAGYFDLKHVIVVDEDVDIHNPTEVEWAVATRFQADRDLVIVPESQGSKLDPSNRDGVGAKMGLDATKPFKAPEMKFKRIYVPGEEAINVADTLKQGGADWRSALKS

SEQ ID NO: 34 - protein, 470 aa, Acidobacteria bacterium RIFCSPLOWO2_02_FULL_68_18
MPKDLRSFLRDLIAARPGEVKRVTTAVDPRFGATAIAERFARDNQYPALYFEKVGASSIPLVLNLTATYDRLAVALETTLRELVPVFGERMTRPVPAREVPRESAPVKEQVWSGDGVDLGKLPLLTHNELDGGPYITSGIGIMRDPESGQVNAGLYRHQVYGRNELGVWFIDGHHGSYIYRRYEERGQPAPIAIAIGHHPAVVMGAVSRLPGIGGEFDAAGGLLGEAVELTRAETCDVPVPGRAEIVIEGEVIPHERRHEGPFGEWPGHYTADGPKPVIRVKTITMRRKPIYYDVFSANREHLVLGSLPRMGSIYRNVKQVVPGLAAVNVPAHSRMHCYLAIAKTRDAEVKKAAFAALNTEPENLKMIVVVDDDINVFNDGDVMWAIGTRFDAARDLLVIPRWSGPGGLLPVGWDYHPDGTRTPRMITATVIDATKPLPPAAYPPRAIVPAAAVDAANVAGITDLQQLPE

SEQ ID NO: 35 - protein, 465 aa, Bacillus sp. FJAT-14578
MKARTFRTWLEHLQTTERLAVIDRKVSLQYEVAKVAKKLDGKKATYFTQVEDYQIPVVSGICSTRADFAEALETDQYQMISKFTEAVASPVPCQLLNNEQAPVKENIILENIDLMKMFPIPVHHEKDSGNYITAGLFIVRDPVTRKQNVSIHRLQISGKNKIGALILPRHTFHLYKQAEEAGRPLECAIVIGVDPVTLLASQASTPFGVDELEIASALRGEPLEVVRCETVDIDVPAYAEIVLEGKILPHVREPEGPFGEFPKYYGPRSDKEVIEITAVTHRNNPIFYTIVPAGYEHLLLGGIPREASLFQSVRQTVPSVTAVHMSVGGTCRYHAIVSIKKRNDGEAKNAILAAFANSFDIKHVVVTDEEVDIFNMEEVEWAIATRFQADKDLVVIHEAQGSKLDPSTREGVGSKMGFDCTVPLNSEPMRYLRVQIPGYNESNVEEYINPHKSIQSVHLEGEKNF

SEQ ID NO: 36 - protein, 464 aa, Thermoflavimicrobium dichotomicum; identical to SEQ ID NO: 24

SEQ ID NO: 37 - protein, 465 aa, Bacillus simplex; identical to SEQ ID NO: 27

SEQ ID NO: 38 - protein, 465 aa, Bacillus sp. FJAT-20673; identical to SEQ ID NO: 26

SEQ ID NO: 39 - protein, 462 aa, Effusibacillus lacus
MKAKTFRTWIEYLQSAGRLAVIDKKVGLQYEVAAIAKKLDGFQAAYFTRVEDYAVPVVSGICSTREDFAEALETDKYGVIPKFTKAVASPIPCRLVEIGQAPVKQNRIREDIDLMRLFPIPVHHEKDSGNYITAGLFIVRDPVTRKQNVSIHRLQVSGKDKLGALLLPRHTFHLYRQAEEAGSPLECAIVIGVDPVTLLASQASTPFGVDELEIAGALRGEPLEVVRCETVDIDVPAFAEIVLEGKILPHVREPEGPFGEFPKYYGPRSDKEVVQITAVTHRNNPIFYTIVPAGYEHLLLGGLPREASLFQTIRQTVPTVKAVHMSPGGTCRYHAIVSIRKRNEGEAKNAIFAALANSFDIKHVVVVDEEVDIFNMEEVEWAIATRFQAERDLVVVHGAQGSKLDPSTNNGVGSKMGLDCTVPLNSEPMRYLRIQIPGYDQVHLDEYINSGASLQPIHLEGA

SEQ ID NO: 40 - protein, 460 aa, Aneurinibacillus terranovensis
MRAKTFRTWMQYLQSKDRLAVIDKNVSLQYEVAAVSKKLDGKKATYFTAVEGYSIPVVSGICSSREDFAEALETDQHGIIPKFTEAVASPTPCRLVDHTEAPVKENIILENINLMKMFPIPVHHEKDSGNYITAGLFIVRDPVTRKQNVSIHRLQVSGKDKLGALILPRHTFHLYKQAEEEGRALECAIVIGVDPVTLLASQASTPYGVDELEIAGSLRGEPLEVVRCETVDIDVPAFAEIVLEGKILPHVREPEGPFGEFPKYYGPRSNKEVVQITAVTHRNNPIFYTIVPAGYEHLLLGGIPREASLFQTIRQTVPSVKAVHMSPGGTCRYHAIVSIKKRNEGEAKNAIIAAFANSFDIKHVVVVDEEVDIFNMEEVEWAIATRFQAEKDLVLVHSSQGSKLDPSTEEGVGSKMGLDCTVPLNSEPMRYLKVYIPGYDEIKVDDYINSEATIKSVHSE

SEQ ID NO: 41 - protein, 464 aa, Paenibacillus naphthalenovorans
MKAKTFRTWIEYLHSKDRLAVVDKKVSLQYEAAAVAKKLDGVKAVYFSHVEDYPIPVVSGICSTREDFAEALETDLNGVIPRFTQAVSSPLPCKPVDRAEAPVKENIIREHIDLMKMLPIPVHHEKDSGNYITAGLFIVRDPVTRKQNVSIHRLQVSGKDKLGALLLPRHTHHLFKQAEEAGRPLECAIVIGVDPVTLLASQASTPFGVDELEIAGALRGEPLEVVRCETVDIDVPAYAEIVLEGQILPHVREPEGPFGEFPKYYGPRSDKEVVQITAVTHRNHPMFYTIVPAGYEHLLLGGIPREASLFQTIRQTVPTVRAVHMSAGGTCRYHAVVSIKKRNEGEAKNAIFAAFANSFDIKHVVVVDEDVDIFNMEEVEWAIATRFQADRDLVVVHGAQGSKLDPSTDEGTGAKMGLDCTIPLQSEPMRYLRVRIPGFEKIELEDYVNSEAGVRSIHLEGKSR

SEQ ID NO: 42 - protein, 462 aa, Domibacillus tundrae
MKAKTFRTWLEHLQKTDRLAVIDRKVSLQYEVAMVAKKLDGKKASYFTNVEEYDVPVVSGICSTREDFAEALGTDQYGVISKFSEAVAAPAPCRLITKQEAPVKENIILEDIDLMKMFPIPVHHDKDSGNYITAGLFIVRDPVTRKQNVSIHRLQISGKNKIGVLLLPRHTFHLYKQAEEAGRPLECAIAIGVDPVTLLASQASTPFGVDELEIASALRDEPLELVRCETVDIDVPACAEIVLEGKILPHIREPEGPFGEFPKYYGPRTDKEVIEITAVNHRNNPIFYTIVAAGYEHLLLGGIPREASLFQSILQTVPSVKAVHMSPGGTCRYHAIVSIKKRNDGEAKNVILAALANSFDIKHVVVTDEDVDIFNMEEVEWAIATRYQADKDLVVIHEAQGSKLDPSTRDGVGSKMGFDCTVPLNSEPMQYLRIRIPGYDECSLDEYINLDKSIQSLHLEKF

SEQ ID NO: 43 - protein, 462 aa, Domibacillus iocasae
MKARTFRTWLEHLQKTDRLAVIDRKVSLQYEVAMVAKKLDGKKASYFTNVEEYDVPVVSGICSTREDFAEALGTDQYGVISKFSEAVAAPAPCRLLTKQEAPVKENIILEDIDLMKMFPIPVHHDKDSGNYITAGLFIVRDPVTRKQNVSIHRLQISGKNKIGVLLLPRHTFHLYKQAEEAGRPLECAIAIGVDPVTLLASQASTPFGVDELEIASALRDEPLELVRCETVDIDVPACAEIVLEGKILPHIREPEGPFGEFPKYYGPRTDKEVIEITAVNHRNNPIFYTIVAAGYEHLLLGGIPREASLFQSILQTVPSVKAVHMSPGGTCRYHAIVSIKKRNDGEAKNVILAALANSFDIKHVVVTDEDVDIFNMEEVEWAIATRYQADKDLVVIHEAQGSKLDPSTRDGVGSKMGFDCTVPLNSEPMQYLRIQIPGYDECSLDEYINPDKSIQSLHLEKF

SEQ ID NO: 44 - protein, 464 aa, Paenibacillus naphthalenovorans
MKAKTFRTWIEYLHSKDRLAVVDKKVSLQYEAAAVAKKLDGVKAVYFSHVEDYPIPVVSGICSTREDFAEALETDLNGVIPRFTQAVSSPLPCKPVDRAEAPVKENIIREHIDLMKMLPIPVHHEKDSGNYITAGLFIVRDPVTRKQNVSIHRLQVSGKDKLGALLLPRHTHYLFKQAEETGRPLECAIVIGVDPVTLLASQASTPFGVDELEIAGALRGEPLEVVRCETVDIDVPAYAEIVLEGQILPHVREPEGPFGEFPKYYGPRSDKEVVQITAVTHRNHPMFYTIVPAGYEHLLLGGIPREASLFQTIRQTVPTVRAVHMSAGGTCRYHAVVSIKKRNEGEAKNAIFAAFANSFDIKHVVVVDEDVDIFNMEEVEWAIATRFQADRDLVVVHGAQGSKLDPSTDEGTGAKMGLDCTIPLQSEPMRYLRVRIPGFEKIELEDYVNSEAGVRSIHLEGKSR

SEQ ID NO: 45 - protein, 468 aa, Brevibacillus thermoruber
MRAKTFRTWLEYLQAQDRLAVIDRPVSLRFELAALAKKLDGRKAVFFSQVDDYQVPVVSGICSTREQFAEALETDPYGLIPKFAQAVASPVPCRQVKKEDAPVKDHIRLDDIDLMRMLPIPVHHEKDAGSYITAGLFIVRDPVTRKQNVSIHRLQVSGKNRLGVLLLPRHTFHLYKQAEEAGRPLECAIAIGVDPVTLLASQASTPFGVDELEIAGALRGEPLEVVRCETVDIDVPAYAEIVLEGRILPNVREPEGPFGEFPKYYGPRSDKEVVEITAVTHRSHPIFYTIVPAGYEHLLLGGIPREASLFQTIRQTVPSVKAVHMSPGGTCRYHAVVSIQKRNEGEGKNAIVAALANSFDIKHVVVVDEDVDIFNMEEVEWAVATRFQAERDLVVVHGAQSSRLDPSTKDGVGSKMGLDCTVPLGSEPMRYLRINIPGYEEIRLEDYLRPGGGTLGETYLERGKKAVR

SEQ ID NO: 46 - protein, 468 aa, Brevibacillus thermoruber; identical to SEQ ID NO: 31

SEQ ID NO: 47 - protein, 449 aa, Brevibacillus sp. OK042
MRAKTLRTWLEYLQSTDRLAVIEKQVHLEYEIAAVAKKLDGRKAAYFTRVEDYRVPVVSGICSTREQFAEALETDQFGLIPKFTDAVNSPLPCRQVQNGEAPVKENIVLKEIDLMRMFPIPVHHEKDSGNYITAGLFIVRDPVTRKQNVSIHRLQVSGKNRLGALLLPRHTFHLYKQAEEAGRPLECAIVIGVDPATLLASQASTPFGVDELEIAGALRNEPLEVVRCETVDIDVPAFAEIVLEGRILPHVREPEGPFGEFPKYYGPKSDKEVVEITAVTHRNNPIFYTIVPAAYEHLLLGGIPREASLFQTVRQTVPSVKAVHMSSGGTCRYHAIVSISKRNEGEAKNAILAALANSFDIKHVVVVDEDVDIYDMEEVEWAIATRFQAERDLVVIHGAQGSKLDPSTNEGVGSKLGFDCTVPLNSEPMRYLRIRIPQYDEIKLEDYLK

SEQ ID NO: 48 - protein, 468 aa, Aeribacillus pallidus; identical to SEQ ID NO: 29

SEQ ID NO: 49 - protein, 468 aa, Brevibacillus sp. WF146; identical to SEQ ID NO: 30

SEQ ID NO: 50 - protein, 472 aa, Bacillus sp. OV194
MKARTFRTWLAHLESTGRLVAIDKKVSLEYEVAAVAKKLDGKKAVYFSNVENYGMPVVSGICSTRDDFAEALETDQYGIIPKFTEAVTSPKACRKVSNQDAPIKENIITENIDLLKTFPIPVHHEMDSGNYITAGLFITRDPVTRKQNVSIHRLQISGKDKLGVLLLPRHTFHLYKQAEEEGRALECAIVVGVDPVTLLASQASTPFGIDELEIASALRSEPLETVRCETVDIDVPAYAEIVLEGKILPHIREPEGPFGEFPKYYGPRSDKEIVQLTAVSHRKDPIFYTIIPAGYEHLLLGGIPREASLIQSVRQTVPSAVAVHMSPGGTCRYHAVVSIKKRNKGEAKNAILAAFANSFDIKHVVVVDEEVDIFNMEEVEWAIATRFQVDKDLVVVEGAQGSKLDPSTNDGFGSKTGFDCTVPLNSEPMRYKRISIPGYANMDIQEYLNETSSGVNPEHLEYGNRRNDSLTS

SEQ ID NO: 51 - protein, 472 aa, Fictibacillus sp. FJAT-27399
MKARTFRTWLAHLESTGRLVAIDKKVSLEYETAAVAKKLDGKKAVYFSNVENYGMPVVSGICSTRDDFAEALETDQYGIIPKFTEAVTSPKACRKVSNQDAPVKENIITENIDLLKTFPIPVHHEMDSGNYITAGLFITRDPVTRKQNVSIHRLQISGKDKLGVLLLPRHTFHLYKQAEEEGRALECAIVVGVDPVTLLASQASTPFGIDELEIASALRSEPLETVRCETVDIDVPAYAEIVLEGKILPHIREPEGPFGEFPKYYGPRSDKEIVQLTAVSHRKDPIFYTIIPAGYEHLLLGGIPREASLIQSVRQTVPSAVAVHMSPGGTCRYHAVVSIKKRNKGEAKNAILAAFANSFDIKHVVVVDEEVDIFNMEEVEWAIATRFQVDKDLVVVEGAQGSKLDPSTNDGFGSKTGFDCTVPLNSEPMRYKRISIPGYANMDIQEYLNETSSGVNPEHLEYGNRRNDSLTS

SEQ ID NO: 52 - protein, 472 aa, Bacillus sp. FJAT-26652
MKARTFRTWLAHLESKGRLVAIDKKVSLEYEIAAVAKKLDGKKAVYFLNVENYGMPVVSGICSTRDDFAEALETDQYGIIPKFTEAVTSPKACRKVSNQDAPVKENIITENIDLLKTFPIPVHHEMDSGNYITAGLFITRDPVTRKQNVSIHRLQISGKDKLGVLLLPRHTFHLYKQAEEEGRALECAIVVGVDPVTLLASQASTPFGIDELEIASALRSEPLETVRCETVDIDVPAYAEIVLEGKILPHIREPEGPFGEFPKYYGPRSDKEIVQLTAVSHRKDPIFYTIIPAGYEHLLLGGIPREASLIQSVRQTVPSAVAVHMSPGGTCRYHAVVSIKKRNKGEAKNAILAAFANSFDIKHVVVVDEEVDIFNMEEVEWAIATRFQVDKDLVVVEGAQGSKLDPSTNDGFGSKTGFDCTVPLNSEPMRYKRISIPGYANMDIQEYLNKTSSGVNPEHLEYGNRRNDSLTS

SEQ ID NO: 53 - protein, 467 aa, Fictibacillus enclensis; identical to SEQ ID NO: 17

SEQ ID NO: 54 - protein, 463 aa, Bacillus kribbensis
MRAKSLRTWLEYLQKTGRLAVIDKEVSLEFEAAALTKKLDGKQAAFFTNVEDYEIPLVSGICSTREQFAEALETDQYEILSKFSRAVASPVECRHVKKEAAPVKENIILDEIDLNIFPIPVHHEKDSGKYITAGLFIVRDPVTRKQNVSIHRLQISGKDKLGVLLLPRHTYHLFKQAEEEGRPLECAIAIGVDPVTLLSSQASTPFGVDELEVASALRNEPLELVRCETVDIDVPAYAEIVLEGKILPKVREPEGPFGEFPKYYGPRGDKEVVQITGITHRNHPIFYTIVPAGYEHLLLGGIPREASLIESVRQTVPSVKAVHMSLGGTCRYHAVISIRKRNEGEGKNTIIAALANSFDIKHVVVVDEEVDVFNMEEVEWAVATRFQAEKDLVVIKGAQGSKLDPSTDEGFGSKMGFDCTIPLDSELMRYQRIHIPGFEEMDIHDYINKDASGLQSQHLEKEI

SEQ ID NO: 55 - protein, 467 aa, Fictibacillus solisalsi; identical to SEQ ID NO: 22

SEQ ID NO: 56 - protein, 463 aa, Bacillus xerothermodurans
MKARTFRTWLEYLHSKNRLAIITRKVSLQYEGAAVTKTMDGKKAVCFTQVEDYPIPVVSGICSTREDFAEALETDQLGIIPKFIEAVASPSPCRSVDSGAAPVKENIILADIDLMQLLPIPVHHEKDSGNYITAGLFIVRDPVTRKQNVSIHRLQVSGKNKLGALLLPRHTYHLYKLAEDAGRALECAIVIGVDPVTLLASQASTPFGVDELELAGALRGEPLEVVRCETVDIDVPAFAEIVLEGRILPHVREPEGPFGEFPKYYGPRSDKEVVHITAVTHRNNPMYYTIVPAGYEHLLLGGIPREASLLQSIRQTVPTVKAVHMSPGGTCRYHAIVSIKKRNEGEGKNAIFAALANSFDIKHVVVVDEEVDIFNMEEVEWAVATRFQAETDLVVVHNALGSKLDPSTAGGVGSKMGLDCTVPLNSEPMRYLRVRIPGYDNMNVEDYVNSRAVFPAKHGEGNN

SEQ ID NO: 57 - protein, 449 aa, Brevibacillus panacihumi
MRRAQTLRTWLEYLRQTERLATIEKQVSLQYELAAVTKKLDGVKAAFFKQVEDYSIPVVSGICSTREQFAEAMETDPYDVIRRFTDAVASPLPCKQVGNDEAPVKENIIREDIDLMRLLPIPVHHEKDAGNYLTAALFIVRDPVTRKQNVSIHRLQVTGKNRLGALLLPRHTYHLYKQAESAGRALECAIVIGVDPLTLLASQASTPFGVDELEIAGALRGEPLEVVRCETVDIDVPAFAEIVLEGKILPHVREPEGPFGEFPKYYGPRSDKEVVEIMAITHRHQPIFYTIVPAGYEHLLLGGIPREASLLQSIKQTVPGVKAVHMSLGGTCRYHAIVSILKRNEGEGKNAILAALANSFDIKHVVVVDEDVDIFNMEEVEWAIATRFQAERDLVVVHGAQGSKLDPSTDEGVGSKLGFDCTIPLDSEPMRYLRVKIPQYEGINLEDYL

SEQ ID NO: 58 - protein, 535 aa, Alicyclobacillus vulcanalis
MSSTTSSPACLTTLVCPCQFRGGENAPFVIIRIQNPLFKSHEQLNSTLFTSIKRVIYIDINSLYDPITRRSQRREANVVAFLSFRAWLEHLRATDRLVEIEAEVSLRYEVAAIAKRLDGRKAVLFHRPEGASIPVTSGLVSHRRWIAEALGCTSNDLIHRFREACERPVPVERVLPDEAPVYARVQTEQIDLMKTLPIPTHHEHDAGPYVTAGLLIVRDPTTRKQNVAIHRLQVTGPDRMGVLLLPRHTLHLFRQAESSGQPLECAVVIGVDPATLLASQASTPFGVDELEIAGALRREPLPVVRCQTVDVDVPADAEIVIEGQILPHVREPEGPFGEFPRYYGPRSDKHVIQVTALLMRENPVYHTIVPAGYEHLLLGGIPREASLFETVRHLVPTVRAVHMTPGGSCRYHAVIAIRKTEEGQGKNAILAAFANSFDIKHVVVVDDDVDIYNPEEVEWAIATRFQAEHDLIVVRHSQGSKLDPSTDDGLGSKMGLDCTIPLDAEPFRFKRISIPGMQTLDWHRYGLSEPSGGEV

SEQ ID NO: 59 - protein, 482 aa, Alicyclobacillus acidocaldarius
MIHKILNSVYDPYHTSQSARREAKVVAFLSFRAWLEHLRETDRLVDVEVEVSLRYEVAAIAKRLDGRKAVMFHRPAGSSVPVASGLVSQRRWIAEALGCAPDDLVHRFREACEQPMPVAHVSEGDAPVYQRVQTEGIDLLKTLPIPTHHEHDAGPYVTAGLVIVRDPATRKQNVAIHRLQVTGPDRMGVLLLPRHTLHLFRQAEAAGRPLECAVAIGVDPATLIASQASTPFGVDELEIASALRRQPLPVVRCRTVDVDVPAEAEIVIEGRILPHVREPEGPFGEFPRYYGPRSDKHVIEVTAVSMRDDPVYHTIVPAGYEHLLLGGIPREASLFETVRQLVPTVRAVHMTPGGSCRYHAVIAIRKTEEGQGKNAILAAFANSFDIKHVVVVDEDVDIYNPEEVEWAIATRFQADRDLVLVEQTQGSKLDPSTDDGLGSKMGLDCTVPLNADPFRFRRITIPGFGEMDWSRYGLTDSFEGKE

SEQ ID NO: 60 - protein, 457 aa, Alicyclobacillus vulcanalis
MAFLSFRAWLEHLRATDRLVEIEAEVSLRYEVAAIAKRLDGRKAVLFHRPEGASIPVTSGLVSHRRWIAEALGCTSNDLIHRFREACERPVPVERVLPDEAPVYARVQTEQIDLMKTLPIPTHHEHDAGPYVTAGLLIVRDPTTRKQNVAIHRLQVTGPDRMGVLLLPRHTLHLFRQAESSGQPLECAVVIGVDPATLLASQASTPFGVDELEIAGALRREPLPVVRCQTVDVDVPADAEIVIEGQILPHVREPEGPFGEFPRYYGPRSDKHVIQVTALLMRENPVYHTIVPAGYEHLLLGGIPREASLFETVRHLVPTVRAVHMTPGGSCRYHAVIAIRKTEEGQGKNAILAAFANSFDIKHVVVVDDDVDIYNPEEVEWAIATRFQAEHDLIVVRHSQGSKLDPSTDDGLGSKMGLDCTIPLDAEPFRFKRISIPGMQTLDWHRYGLSEPSGGEV

SEQ ID NO: 61 - protein, 473 aa, Alicyclobacillus mali
MIHITRRNRRVRGANAVAFLSFRAWLQHLRNTDRLLDVEVEVSLRYEVAAIAKRVDGQKAVLFHRTDGAPVPVASGLVSHRRWIAEALGCGQVDLVHKFREACEQPVPVVRVMETEAPVYHQVQVDEIDLLKTLPIPTHHEHDAGPYITAGLVIVRDPSTRKQNVAIHRLQVTGPNRMGVLLLPRHTLHLFRQAEAAGRPLECAVVIGVDPATLLASQASTPFGVDELEIASALRGEPLPVVRCRTVDVDVPAEAEIVIEGRILPDVREPEGPFGEFPRYYGPRSDKHVIEVTGVLMRENPVYHTIVPAGYEHLLLGGIPREASLFETVRQLVPTVRAVHMTPGGSCRYHAVISIRKTEEGQGKNAILAALANSFDIKHVVVVDQDVDIYNPEEVEWAIATRFQADRDLVLVSNSQGSKLDPSTDDGVGSKMGLDCTVPLNADSFRFRRIAIPGFAEMDWSRYGLKNSFEEGE

SEQ ID NO: 62 - protein, 474 aa, Bacillus ligniniphilus
MQRATSLRTWLKYLEEKNEVAIIDKNVSLNHEIAAITKKLDGHKAAFFTNVENYRTPVIAGTATTRQQFAEALNINEQELHERFLAAMDAPLPCKEVESKDAPVHENVILGDDVDLMKLFPIPVHHEKDSGNYIAAGLSIAKDPATGNQNVSIHRLQVSGKNRLGALILPRHLYNFYKNNEEQGKPLDIAIVIGVDPVKLLASQAIAPLGQDEMEIAGALYGESVPVVKCKTIDVNVPAYAEIVLEGRILPGVREPEGPFGEFPKYYGPKSNKEVIEITAITHRNNPIFQTIVPASYEHLLLGGIPREASLLNTLRDTVPTVRDVHLTLGGTCRYHLVVSMKKRNEGEGKNVILSAFAGHYDVKHVVVVDEEVDIYNLEEVEWALATRFQADKDLVVINHALGSKLDPSTDDGVSSKVGFDCTVPLSSKVSINNEPQEFERISIPGLERVNETEYLLQQVQVQEEHKIYFHEEK

SEQ ID NO: 63 - protein, 468 aa, Jeotgalibacillus soli
MQKNKSLRSWLGHLKELNQLAVVDKKVSLEFEIAGIVKKLDGTKAAYFPNVKGYQIPVVAGTATTRRQFADALDITETQLNEKFLDALEHPIPCKEIPSEAAPVHENVILGEAVDLLKYFPIPIHHEKDSGNYISAGLSIAKDPISGSQNVSIHRLQVSGKNKLGALILPRHLYSFYKEAEEKGQPLNVAIVIGVDPVTILASQAIAPLGQDEMEITGALKGRPEPVVKCKTIDVLVPAHAEIVLEGRILPIVREPEGPFGEFPKYYGPRSDKEVIEITAITHRNNPIYQTIVAASYEHLLLGGIPREASLLQALRNTVPTVKDVHLTLGGTCRYHLVVQMKKRNEGEAKNVILAAFAGHYDVKHVVVVDDDVDIYNLEEVEWAIATRFQAEKDLVVINGALGSKLDPSTDNGVGSKLGFDCTVPLNSEPMEFEKIQIPGMKGLDTEYYRNQDVRSIEVVSEHFIERD

SEQ ID NO: 64 - protein, 464 aa, Cohnella sp. 6021052837
MKSRTLRTWIRHMDESGQLATVDKPVSTRFEISAFTKQYDGSKAVFFPQVEGYGMAVVSGIASTRAQFAQALGCSENEMIPKFLDAIDNPLPSREVAEEEAPVQEIVIDKDIDLTSLFPISLHQEKDSGPYITAGLAIVRDPDTGIQNVSIHRLQVSGPNRLGALLLPRHTYNAFKKKEEKGEPLEIAIVLGVDPVLMLASQAIAPLGQDELEIAGALKGEPVPVVKCKTIDVDVPADAEIVLEGKIMPHVREPEGPFGEFPAYYGLQSDKEVIEIGCVTHRKDPIYQTCLAGSDENLLLGAIPREASLLRSMQNTVSTIRNVHLTMGGKCRFHLVVSMKKRNEGEAKNAIFAAFAGHYDVKKVIVVDEEIDIFDSDMVDWCVATRFQADKDLVVVHGALGSKLDPSTDNGVGSKLGFDCTVPLDAEPMDYEVTVIPGMKEFIAKGKVLERLPDETAKRYLQTD

SEQ ID NO: 65 - protein, 464 aa, Paenibacillus sp. 32352
MIANSLRSWLQHMNESGQLAVVDKHVSTEFEIAALTKQYDGVKPVFFPKVSGYTTSAVAGIASTRSHFAQALGCKEEEMIPKFLEAIDHPLPTEPVSSEEAPVHEVVIDTDIDLLKLFPIPIHQEKDSGPYISAGLAIVRDPETGNQNVSIHRLQVSGPNRLGALILPRHTFNFYKKMEEKGLPLEIAIVIGVDPILMLASQAIAPLGQDELEIAGALKNAPIPVVRCKTVNIDVPAHAEIVLEGKILPKVREPEGPFGEFPAYYGAKSNKEVIEIHCVTHRKDPIYQTCLAGSDENLLLGGIPREASLLRSLKNTVPTVRNVHLTKGGKCRFHMVVSMKKRNEGEAKNVILAAFAGHYDVKKVIVVDEEIDIFDMDQVDWCLATRFQAEHDLVVVHRALGSKLDPSTDNGVGSKMGFDCTVPLSAAPNDYEVTRIPNFAEYVQNAVVTDRLPQQIVDRYFGNN

SEQ ID NO: 66 - protein, 465 aa, Paenibacillus sp. UNC451MF
MKANSLRSWLQFMNESDQLAVVDKHVSTEFELAALTKQYDGVKPVFFPKISGYTTSAVAGIASTRSHFAQAMGCKENEMIPKFLEAIDHPLPTELVSSEEAPVHEVVINTDIDLLSLFPIPIHQEKDSGPYISAGLAIVRDPESGSQNVSIHRLQVSGKNRLGALILPRHTFNFYKKMEEKGLPLEIAIVIGVDPILMLASQAIAPLGQDEMEIAGALKNAPIPVVRCKTVNIDVPVHAEIVLEGKILPKIREPEGPFGEFPAYYGAQSDKEVIEVHCVTHRKDPIYQTCLAGSDENLLLGGIPREASLLRSLKNTVSTVRNVHLTKGGKCRFHMVVSMKKRNEGEAKNVILAAFAGHYDVKKVIVVDEEIDIFDMDQVDWCVATRFQAEHDLVVVHRALGSKLDPSTDNGVGSKIGFDCTVPLSAAPFDYEVTRIPNLAEYVQNATVVDKLPQQFVDRYFPGNK

SEQ ID NO: 67 - protein, 447 aa, Desulfotomaculum nigrificans; identical to SEQ ID NO: 6

SEQ ID NO: 68 - protein, 447 aa, Desulfotomaculum nigrificans; identical to SEQ ID NO: 7

SEQ ID NO: 69 - protein, 450 aa, Desulfotomaculum putei; identical to SEQ ID NO: 18

SEQ ID NO: 70 - protein, 474 aa, Rhodoplanes sp. Z2-YC6860; identical to SEQ ID NO: 32

SEQ ID NO: 71 - protein, 479 aa, Rhodoplanes sp. Z2-YC6860; identical to SEQ ID NO: 33

SEQ ID NO: 72 - protein, 443 aa, Caloramator mitchellensis
MERQMLRFELERLRQMGLLLETDVEVDAKFELGGILKKLKNKRPVLFNRVKGYKMPVCAALYTDREAVYKLSNISHEDRIYKLMDAIANPKPFKVVDRGPVMENIITGNIDIRKFPHPYFHEKDSSTYITAGMLVVKDIKTGRRYTSIRRFQVIDRNKISALIASPYLINQFLEMEKMNRPLEVAIVIGYDVPFLLASQVSSELYGVDKYEIDSALRGEPLEVVKCKTIDLEVPAHAEIVLEGVMVPNKREMEGPFGELMGYYGEQKMHPYIEIKAIMHRNNPIYQAAFPCREEHLANGLLREVELYYALKRLVDVKDVHVTLSGGYRFHAIASINKKQEGDGKTAIIGALSSIKDLKHVVIVDDDIDIFDNEDVELAIATRFQSSRDLVLIPEACGSGLDPSHTTRGVSDKMGLDATRPLNNERFERCRIPGLENIDLRKYF

SEQ ID NO: 73 - protein, 499 aa, Paraburkholderia tropica
MSEPTLTAANVTDLRSAIELLKQHEGEYVETNEPVDPEAELSGVYRYVGAGGTTARPTKRGGPAMMFNNVKGFPAFRVAIGMLGSRKRVARLLGAEPEKLGFLLKDSVQHPIEPVLAEPGRAFCQEIVHRADEPGFDLRTLLPAPTNTPEDAGPYLTMGLCYAADPETGEHDVTIHRLCVQSKDELTMWLTPGRHIDAFRMKAEAAGKPLPISVSIGVDPAIYVAACFEPPTTPLGYDELKIAGALRGRPVELSQCLTINARAIAHAEIVIEGELLPNVRQREDQNTDTGKAMPEFPGYTGEAKDELPVIKVKAVTHRLNPILQTTMGPGEEHVNMAGIPTEASILQMVERAMPGKLLNVYAHNSGGGKLLAVMQFKKSAAVDEGRQRQAAILAFAAFPELKHVMLVDEDVDIFDTDDVLWAMQTRFQGDVDTINIPGVRCHPLDPSQTPAFSPSIPEVGTSCKTIFDCTVPFKLKHEFERSKFLEVDVNRFLPGFNGN

SEQ ID NO: 74 - protein, 500 aa, Asperilligus niger
MSAQPAHLCFRSFVEALKVDNDLVEINTPIDPNLEAAAITRRVCETNDKAPLFNNLIGMKNGLFRILGAPGSLRKSSADRYGRLARHLALPPTASMREILDKMLSASDMPPIPPTIVPTGPCKENSLDDSEFDLTELPVPLIHKSDGGKYIQTYGMHIVQSPDGTWTNWSIARAMVHDKNHLTGLVIPPQHIWQIHQMWKKEGRSDVPWALAFGVPPAAIMASSMPIPDGVTEAGYVGAMTGSSLELVKCDTNDLYVPATSEIVLEGTLSISETGPEGPFGEMHGYIFPGDTHLGAKYKVNRITYRNNAIMPMSSCGRLTDETHTMIGSLAAAEIRKLCQQNDLPITDAFAPFESQVTWVALRVDTEKLRAMKTTSEGFRKRVGDVVFNHKAGYTIHRLVLVGDDIDVYEGKDVLWAFSTRCRPGMDETLFEDVRGFPLIPYMGHGNGPAHRGGKVVSDALMPTEYTTGRNWEAADFNQSYPEDLKQKVLDNWTKMGFSN

SEQ ID NO: 75 - protein, 503 aa, Saccharomyces cerevisiae
MRKLNPALEFRDFIQVLKDEDDLIEITEEIDPNLEVGAIMRKAYESHLPAPLFKNLKGASKDLFSILGCPAGLRSKEKGDHGRIAHHLGLDPKTTIKEIIDYLLECKEKEPLPPITVPVSSAPCKTHILSEEKIHLQSLPTPYLHVSDGGKYLQTYGMWILQTPDKKWTNWSIARGMVVDDKHITGLVIKPQHIRQIADSWAAIGKANEIPFALCFGVPPAAILVSSMPIPEGVSESDYVGAILGESVPVVKCETNDLMVPATSEMVFEGTLSLTDTHLEGPFGEMHGYVFKSQGHPCPLYTVKAMSYRDNAILPVSNPGLCTDETHTLIGSLVATEAKELAIESGLPILDAFMPYEAQALWLILKVDLKGLQALKTTPEEFCKKVGDIYFRTKVGFIVHEIILVADDIDIFNFKEVIWAYVTRHTPVADQMAFDDVTSFPLAPFVSQSSRSKTMKGGKCVTNCIFRQQYERSFDYITCNFEKGYPKGLVDKVNENWKRYGYK

SEQ ID NO: 76 - protein, 513 aa, Candida dubliniensis
MSLNPALKFRDFIQVLKNEGDLIEIDTEVDPNLEVGAITRKAYENKLAAPLFNNLKQDPENIDPKNLFRILGCPGGLRGFGNDHARIALHLGLDSQTPMKEIIDFLVANRNPKKYIPPVLVPNDQSPHKKHHLTKEQIDLTKLPVPLLHHGDGGKFIQTYGMWVLQTPDKSWTNWSIARGMVHDSKSITGLVINPQHVKQVSDAWVAAGKGDKIPFALCFGVPPAAILVSSMPIPDGATEAEYIGGLCNQAVPVVKCETNDLEVPADCEMVFEGYLDRDTLVREGPFGEMHGYCFPKDHHTQPLYRVNHISYRDQAIMPISNPGLCTDETHTLIGGLVSAETKYLISQHPVLSKIVEDVFTPYEAQALWLAVKINTHELVKLKTNAKELSNLVGDFLFRSKECYKVCSILHEIILVGDDIDIFDFKQLIWAYTTRHTPVQDQLYFDDVKPFALAPFASQGPLIKTRQGGKCVTTCIFPKQFTDPDFEFVTCNFNGYPEEVKNKISQNWDKYYK

SEQ ID NO: 77 - protein, 497 aa, Escherichia coli
MDAMKYNDLRDFLTLLEQQGELKRITLPVDPHLEITEIADRTLRAGGPALLFENPKGYSMPVLCNLFGTPKRVAMGMGQEDVSALREVGKLLAFLKEPEPPKGFRDLFDKLPQFKQVLNMPTKRLRGAPCQQKIVSGDDVDLNRIPIMTCWPEDAAPLITWGLTVTRGPHKERQNLGIYRQQLIGKNKLIMRWLSHRGGALDYQEWCAAHPGERFPVSVALGADPATILGAVTPVPDTLSEYAFAGLLRGTKTEVVKCISNDLEVPASAEIVLEGYIEQGETAPEGPYGDHTGYYNEVDSFPVFTVTHITQREDAIYHSTYTGRPPDEPAVLGVALNEVFVPILQKQFPEIVDFYLPPEGCSYRLAVVTIKKQYAGHAKRVMMGVWSFLRQFMYTKFVIVCDDDVNARDWNDVIWAITTRMDPARDTVLVENTPIDYLDFASPVSGLGSKMGLDATNKWPGETQREWGRPIKKDPDVVAHIDAIWDELAIFNNGKSA

SEQ ID NO: 78 - protein, 495 aa, Enterobacter cloacae
MQNPINDLRSAIALLQRHPGHYIETDHPVDPNAELAGVYRHIGAGGTVKRPTRTGPAMMFNSVKGYPGSRILVGMHASRERAALLLGCVPSKLAQHVGQAVKNPVAPVVVPASQAPCQEQVFYADDPDFDLRKLLPAPTNTPIDAGPFFCLGLVLASDPEDTSLTDVTIHRLCVQERDELSMFLAAGRHIEVFRKKAEAAGKPLPVTINMGLDPAIYIGACFEAPTTPFGYNELGVAGALRQQPVELVQGVAVKEKAIARAEIIIEGELLPGVRVREDQHTNTGHAMPEFPGYCGEANPSLPVIKVKAVTMRNHAILQTLVGPGEEHTTLAGLPTEASIRNAVEEAIPGFLQNVYAHTAGGGKFLGILQVKKRQPSDEGRQGQAALIALATYSELKNIILVDEDVDIFDSDDILWAMTTRMQGDVSITTLPGIRGHQLDPSQSPDYSTSIRGNGISCKTIFDCTVPWALKARFERAPFMEVDPTPWAPELFSDKK

SEQ ID NO: 79 - protein, 502 aa, Klebsiella pneumoniae
MTAPIQDLRDAIALLQQHDNQYLETDHPVDPNAELAGVYRHIGAGGTVKRPTRIGPAMMFNNIKGYPHSRILVGMHASRQRAALLLGCEASQLALEVGKAVKKPVAPVVVPASSAPCQEQIFLADDPDFDLRTLLPAPTNTPIDAGPFFCLGLALASDPVDASLTDVTIHRLCVQGRDELSMFLAAGRHIEVFRQKAEAAGKPLPITINMGLDPAIYIGACFEAPTTPFGYNELGVAGALRQRPVELVQGVSVPEKAIARAEIVIEGELLPGVRVREDQHTNSGHAMPEFPGYCGGANPSLPVIKVKAVTMRNNAILQTLVGPGEEHTTLAGLPTEASIWNAVEAAIPGFLQNVYAHTAGGGKFLGILQVKKRQPADEGRQGQAALLALATYSELKNIILVDEDVDIFDSDDILWAMTTRMQGDVSITTIPGIRGHQLDPSQTPEYSPSIRGNGISCKTIFDCTVPWALKSHFERAPFADVDPRPFAPEYFARLEKNQGSAK

(19) United States
(12) Patent Application Publication
(10) Pub. No.: US 2021/0180095 A1
(43) Pub. Date: Jun. 17, 2021

(54) METHODS OF PRODUCING 2,5-FURANDICARBOXYLIC ACID

(71) Applicant: SHELL OIL COMPANY, HOUSTON, TX (US)

(72) Inventors: Karl Alex Peter PAYNE, Manchester (GB); David LEYS, Manchester (GB); David Alexander PARKER, Houston, TX (US); Stephen Andrew MARSHALL, Manchester (GB)

(21) Appl. No.: 16/762,336

(22) PCT Filed: Nov. 9, 2018
(86) PCT No.: PCT/US2018/060061

§ 371 Date: May 7, 2020

Related U.S. Application Data
(60) Provisional application No. 62/584,179, filed on Nov. 10, 2017.

(51) Int. Cl. C12P 17/04; C12N 9/88

(52) U.S. Cl. CPC C12P 17/04; C12N 9/88

(57) ABSTRACT

Method for preparing a 2,5-furandicarboxylic acid (“FDCA”) comprising: contacting a polypeptide comprising an amino acid sequence that has greater than 34% sequence identity with an amino acid sequence set out in SEQ ID NO:1 or SEQ ID NO:2 with furoic acid in the presence of carbon dioxide; wherein the polypeptide has carboxylase and decarboxylase activity and comprises (i) the amino acid corresponding to H297 or a functional substitution thereof and (ii) at least one of (a) the amino acid corresponding to R305 or a functional substitution thereof; and (b) the amino acid corresponding to R332 or a functional substitution thereof; wherein the position is numbered relative to the amino acid sequence of SEQ ID NO:1 or SEQ ID NO:2.

### CROSS REFERENCE TO RELATED APPLICATIONS

This application claims the benefit of U.S. Provisional Application No. 62/584,179, filed Nov. 10, 2017, which is incorporated herein by reference.

### REFERENCE TO A “SEQUENCE LISTING”

The Sequence Listing submitted herewith is hereby incorporated by reference in its entirety.

### TECHNICAL FIELD

Embodiments described herein relate methods of producing 2,5-furandicarboxylic acid (“FDCA”), particularly from furoic acid.

### BACKGROUND

With the diminishing supply of crude mineral oil, use of renewable energy sources is becoming increasingly important to produce liquid fuels and/or chemicals. These fuels and/or chemicals from renewable energy sources are often referred to as biofuels. Biofuels and/or biochemicals derived from non-edible renewable energy sources are preferred as these do not compete with food production.

There have been various investigations into methods of producing 2,5-furandicarboxylic acid (“FDCA”), a furan derivative that is also known as dehydromucic acid, because the utilization value of FDCA as an intermediate in each of various fields including bioplastic monomers, drugs, agricultural chemicals, pesticides, antibacterial agents, flavors, and polymer materials is high. Moreover, its value extends further because it can be derived from biological sources serve as a potential replacement for petrochemical derived monomers, such as terephthalate, that are used in polymers such as polyethylene terephthalate (PET) plastics.

For these and other reasons, FDCA was identified as one of the Top-12 priority chemicals in the DOE report on Top Value-Added Chemicals from Biomass (Top Value-Added Chemicals from Biomass, Volume I—Results of screening for potential Candidates from Sugars and Synthesis gas, Department of Energy (USA), 2004). The DOE report discloses, on page 27, some potential utilities for FDCA. These include a role as substrate for the production of succinic acid, 2,5-bis(aminomethyl)-tetrahydrofuran, 2,5-dihydroxymethyl-tetrahydrofuran, 2,5-dihydroxymethylfuran and SEQ ID NO:2,5-furandicarbaldehyde. The production of FDCA by chemical oxidative dehydration of C6 sugars are well known, such as those disclosed in the DOE report, U.S. Pat. No. 3,326,944, and WO2011043660. These chemical processes suffer from certain challenges and technical barriers such as those indicated in table 13 on page 26 of the DOE report. The position of biotransformation—possibly enzymatic conversions to or production of FDCA was unknown at the time the DOE report published.

Although enzymatic production processes of FDCA have been developed, they also suffer from certain challenges, including the use of hydroxymethylfurfural (HMF) or related compounds such as HMF alcohol (2,5-dihydroxymethyl furan) and HMF acid (5-hydroxymethyl-2-furancarboxylic acid). For instance, WO2009023174 discloses a method of converting hydroxymethylfurfural into FDCA by contacting the hydroxymethylfurfural species in a mixture with a chloroperoxidase while controlling hydrogen peroxide in the mixture. WO2011026913 discloses an oxidoreductase that converts HMF into FDCA. Similarly, WO2012064195 discloses a genetically modified cell that converts HMF acid to FDCA. There are challenges associated with the use of HMF or related compounds because HMF production from biomass material, such as cellulosic material, has only been achieved at pilot plant scale, which present hurdles for commercial scale production of chemicals from biomass. As such, there is still a need for enzymatic production of FDCA from other precursor compounds.

### SUMMARY

Embodiments of the present disclosure allows for enzymatic production of FDCA from furoic acid, which may be produced from furfural: a compound that is readily produced as an unwanted byproduct of acid catalyzed thermo hydrolysis of hemicellulosic material, a process that is already carried out on industrial scale. See Moreau, C., Belgacem, M. N., and Gandini, A. (2004) Recent catalytic advances in the chemistry of substituted furans from carbohydrates and in the ensuing polmers. Top Catal 27, 11-30.

The present disclosure provides a method for preparing a 2,5-furandicarboxylic acid (“FDCA”) comprising: contacting a HmfF polypeptide comprising an amino acid sequence that has greater than 34% sequence identity with an amino acid sequence set out in SEQ ID NO:1 or SEQ ID NO:2 with furoic acid in the presence of carbon dioxide, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2. Optionally, the method is performed in vitro, such as a cell-free lysate system.

The present disclosure also provides for a vector or plasmid or isolated and purified polynucleotide comprising a nucleic acid sequence encoding a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

The present disclosure also provides for a recombinant host cell comprising a nucleic acid sequence encoding a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

Further, the present disclosure provides for a cell-free lysate composition comprising:
a. a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2;
b. furoic acid; and
c. carbon dioxide.

In addition, the present disclosure provides a method comprising:
d. providing a reactor having a pressure of greater than 1 bar, said reactor comprising furoic acid, carbon dioxide in an amount greater than that at ambient conditions, such as at least 400 ppm, a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2, and
e. allowing the HmfF polypeptide, furoic acid, and carbon dioxide to remain in the reactor for a period of time to allow production of FDCA.

Optionally, for any one of the various aspects of the present disclosure noted above, the functional substitution can comprise a conservative substitution. Optionally, the conservative substitution can comprise at least one of (i) the amino acid corresponding to R305K, and (ii) the amino acid corresponding to R332K, wherein the position is numbered relative to the amino acid sequence of SEQ ID NO:1 or SEQ ID NO:2. Optionally, for any one of the various aspects of the present disclosure noted above, the carbon dioxide can be present in an amount greater than ambient conditions, such as at least 400 ppm, at least 600 ppm, at least 800 ppm, or at least 1000 ppm. Optionally, at least a portion of the carbon dioxide may be under supercritical conditions. Optionally, for any one of the various aspects of the present disclosure noted above, the reactor, reaction, composition, or contacting step, respectively, can have a pH in a range of 5-9, preferably 6-8. Optionally, for any one of the various aspects of the present disclosure noted above, the furoic acid can be present in an amount of at least 0.1 mM, at least 0.5 mM, at least 1 mM, at least 5 mM, at least 10 mM, at least 50 mM, at least 0.1M, or at least 0.5M. It is understood that the furoic acid may be provided in a solution and/or it may be provided as part of a mixture containing biomass. Optionally, for any one of the various aspects of the present disclosure noted above, the reactor, reaction, composition, or contacting step, respectively, can be conducted at a temperature suitable for the HmfF polypeptide to catalyze the reaction, such as a temperature in a range of 35-60 degrees C., including 45-55 degrees C. Optionally, for any one of the various aspects of the present disclosure noted above, the reactor, reaction, composition, or contacting step, respectively, may be under pressure, such as greater than 1 bar or greater than atmospheric or ambient pressure to drive the transformation of furoic acid to at least FDCA. Optionally, the pressure can be in greater than 2, 3, 4, 5, or 10 bar.

Other features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Embodiments of the invention may be better understood by reference to the drawing in combination with the detailed description of specific embodiments presented herein.

FIG. 1 illustrates a reaction scheme of the carboxylation and decarboxylation reactions that can take place in methods described in the present disclosure;

FIG. 2 shows decarboxylation activity assays of a HmfF as described in the present disclosure monitored by UV-vis (panel B); enzymatic activity in light/dark anaerobic/aerobic conditions (panel C); michaelis menten characterization (panel D), enzymatic activity in varying pH conditions (panel E); and enzymatic activity in varying temperature conditions (panel F);

FIG. 3 is an HPLC chromatogram demonstrating production of FDCA via a method as described in the present disclosure;

FIG. 4 is a mass spectrum demonstrating production of FDCA via a method as described in the present disclosure; and

FIG. 5 depicts the binding site portion of the P. thermopropionicum HmfF crystal structure in complex with FMN (flavin mononucleotide; an analogue of prFMN, prenylated FMN) (to 2.7 Å resolution).

FIG. 6 depicts a proposed mechanism for the HmfF (de)carboxylation reaction.

FIG. 7. A bar chart of k_{cat} values for the P. thermopropionicum HmfF and selected variants with FDCA and PDCA substrates.

### DETAILED DESCRIPTION

Throughout the present specification and the accompanying claims, the words “comprise” and “include” and variations such as “comprises,” “comprising,” “includes,” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

The term “polynucleotide” includes poly deoxyribonucleic acids (DNA) and poly ribonucleic acids (RNA) and the term may refer to either DNA or RNA. The skilled person will be aware of the differences in stability of DNA and RNA molecules. Thus, the skilled person will be able to understand from the context of the use of the term “polynucleotide” which of the forms of polynucleotide (DNA and/or RNA) is suitable.

The term “sequence identity” is known to the skilled person. Sequence identity between amino acid sequences or between nucleic acid sequences can be determined by comparing an alignment of the sequences using various methods known to one of ordinary skill. To determine the degree of sequence identity shared by two amino acid sequences or by two nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The degree of identity shared between sequences is typically expressed in terms of percentage identity between the two sequences and is a function of the number of identical positions shared by identical residues in the sequences (i.e., % identity=number of identical residues at corresponding positions/total number of positions×100).

When comparing the level of sequence identity to, for example, SEQ ID NO:1 or SEQ ID NO:2, this suitably can be done relative to the whole length of SEQ ID NO:1 or SEQ ID NO:2 (i.e., a global alignment method is used), to avoid short regions of high identity overlap resulting in a high overall assessment of identity. For example, a short polypeptide fragment having, for example, five amino acids might have a 100% identical sequence to a five amino acid region within the whole of SEQ ID NO:1, 2, 3, or 4, but this does not provide a 100% amino acid identity according to the present definitions, unless the fragment forms part of a longer sequence which also has identical amino acids at other positions equivalent to positions in SEQ ID NO:1 or SEQ ID NO:2. As mentioned above, sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

The skilled person will be aware of the fact that several different computer programs, using different mathematical algorithms, are available to determine the identity between two sequences. One computer program option to determine the percent identity between two nucleotide sequences is the GAP program in the Accelrys GCG software package (Accelerys Inc., San Diego U.S.A.). Substitution matrices that may be used are for example a BLOSUM 62 matrix or a PAM250 matrix, with a gap weight of 16, 14, 12, 10, 8, 6, or 4 and a length weight of 1, 2, 3, 4, 5, or 6. If the GAP program in the Accelrys GCG software package (Accelerys Inc., San Diego U.S.A) is used to determine the percent identity between two nucleotide sequences, a NWSgapdna CMP matrix and a gap weight of 40, 50, 60, 70, or 80 and a length weight of 1, 2, 3, 4, 5, or 6 may be used. The skilled person will appreciate that all these different parameters will yield slightly different results but that the overall percentage identity of two sequences is not significantly altered when using different algorithms

Another option to determine the percent identity of two amino acid or nucleotide sequences can be the algorithm of E. Meyers and W. Miller (Meyers et al. (1989)) which has been incorporated into the ALIGN program (version 2.0) (available at the ALIGN Query using sequence data of the Genestream server IGH Montpellier France http://vegajgh.mrs.fr/bin align-guess.cgi) using a PAM120 weight residue table, a gap length penalty of 12 and a gap penalty of 4.

Yet another option to determine the percentage sequence identity can be the Needleman-Wunsch Global Sequence Alignment tool, using default parameter settings (such as, for protein alignment, Gap costs Existence:11 Extension:1). The Needleman-Wunsch algorithm was published in J. Mol. Biol. (1970) vol. 48:443-53. The Needleman-Wunsch Global Sequence Alignment Tool available from the National Center for Biotechnology Information (NCBI), Bethesda, Md., USA, for example via http://blast.ncbi.nlm.nih.gov/Blast.cgi.

Optionally, a preferred method to determine the percentage identity and/or similarity between nucleotide or amino acid sequences is BLAST (Basic Local Alignment Search Tool). Queries using the BLASTn, BLASTp, BLASTx, tBLASTn and tBLASTx programs of Altschul et al. (1990) may be posted via the online versions of BLAST made accessible by the National Center for Biotechnology Information (NCBI) of the National Institute of Health (NIH). Alternatively, a standalone version of BLAST {e.g., version 2.2.24 (released 23 Aug. 2010)) downloadable also via the NCBI internet site may be used.

For amino acid sequences the relevant functional properties are the physico-chemical properties of the amino acids. A “conservative substitution” is a change at a specific location of an amino acid or nucleotide sequence that are likely to preserve the functional properties of the original residue as if no change occurred. A conservative substitution for an amino acid in a polypeptide of the invention may be selected from other members of the class to which the amino acid belongs.

For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. The characteristics of amino acids can be summarized as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H |

Conservative substitutions include, for example, Lys for Arg and vice versa to maintain a positive charge; Glu for Asp and vice versa to maintain a negative charge; Ser for Thr so that a free —OH is maintained; and Gin for Asn to maintain a free —H₂.

For instance, conservative amino acid substitution can be a substitution such as the conservative substitutions shown below. The substitutions shown are based on amino acid physical-chemical properties, and as such, are independent of organism.

| Original | Conservative | Exemplary |
|---|---|---|
| Residue | Substitutions | Substitutions |
| Ala (A) | val; leu; ile | Val |
| Arg (R) | lys; gln; asn | Lys |
| Asn (N) | gln; his; lys; arg | Gln |
| Asp (D) | Glu | Glu |
| Cys (C) | Ser | Ser |
| Gln (Q) | Asn | Asn |
| Glu (E) | Asp | Asp |
| Gly (G) | pro; ala | Ala |
| His (H) | asn; gln; lys; arg | Arg |
| Ile (I) | leu; val; met; ala; phe | Leu |
| Leu (L) | ile; val; met; ala; phe | Ile |
| Lys (K) | arg; gln; asn | Arg |
| Met (M) | leu; phe; ile | Leu |
| Phe (F) | leu; val; ile; ala; tyr | Leu |
| Pro (P) | Ala | Ala |
| Ser (S) | Thr | Thr |
| Thr (T) | Ser | Ser |
| Trp (W) | tyr; phe | Tyr |
| Tyr (Y) | trp; phe; thr; ser | Phe |
| Val (V) | ile; leu; met; phe; ala | Leu |

For nucleotide sequences the relevant functional properties is mainly the biological information that a certain nucleotide carries within the open reading frame of the sequence in relation to the transcription and/or translation machinery. It is common knowledge that the genetic code has degeneracy (or redundancy) and that multiple codons may carry the same information in respect of the amino acid for which they code. For example, in certain species the amino acid leucine is coded by UUA, UUG, CUU, CUC, CUA, CUG codons (or TTA, TTG, CTT, CTC, CTA, CTG for DNA), and the amino acid serine is specified by UCA, UCG, UCC, UCU, AGU, AGC (or TCA, TCG, TCC, TCT, AGT, AGC for DNA). Nucleotide changes that do not alter the translated information are considered conservative changes.

As used herein, a “vector” is a polynucleotide construct for introducing a polynucleotide sequence into a cell. In some embodiments, the vector comprises a suitable control sequence operably linked to and capable of effecting the expression of the polypeptide encoded in the polynucleotide sequence in a suitable host. An “expression vector” has a promoter sequence operably linked to the polynucleotide sequence (e.g., transgene) to drive expression in a host cell, and in some embodiments a transcription terminator sequence. In some embodiments, the vectors are deletion vectors. In some embodiments, vectors comprise polynucleotide sequences that produce small interfering RNA or antisense RNA transcripts that interfere with the translation of a target polynucleotide sequence.

Unless otherwise defined herein, scientific and technical terms used herein will have the meanings that are commonly understood by those of ordinary skill in the art. Generally, nomenclatures used in connection with techniques of biochemistry, enzymology, molecular and cellular biology, microbiology, genetics and protein and nucleic acid chemistry and hybridization, described herein, are those well known and commonly used in the art.

General molecular biological techniques such as hybridization experiments, PCR experiments, restriction enzyme digestions, transformation of hosts etcetera may be performed according to the standard practice known to the skilled person as disclosed in Sambrook et al, 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

The present disclosure provides a method for preparing a 2,5-furandicarboxylic acid (“FDCA”) comprising: contacting a polypeptide comprising an amino acid sequence that has greater than 34%, optionally at least 40%, optionally at least 50%, optionally at least 60%, optionally at least 70%, optionally at least 80%, optionally at least 90%, or optionally at least 91%, 92%, 93%, 94%, 95%, 96%, 97%, 98%, or 99% sequence identity with an amino acid sequence set out in SEQ ID NO:1 or SEQ ID NO:2 with a furoic acid in the presence of carbon dioxide, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

A polypeptide that has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2 may be referred to as an “HmfF polypeptide” or “HmfF enzyme,” unless otherwise indicated. For instance, the polypeptide comprising an amino acid sequence of SEQ ID NO:1 is an example of an HmfF enzyme, which is from the thermophilic bacterium Pelotomaculum thermopropionicum. Another example of an HmfF polypeptide is the polypeptide comprising an amino acid sequence of SEQ ID NO:2, which is from the bacterium Geobacillus kaustophilus. The sequence identity between SEQ ID NO:1 and SEQ ID NO:2 is about 55%.

Optionally, the carbon dioxide can be present in an amount greater than ambient conditions, such as at least 400 ppm, at least 600 ppm, at least 800 ppm, or at least 1000 ppm. Optionally, at least a portion of the carbon dioxide may be under supercritical conditions. Optionally, the contacting step can take place at a pH in a range of 5-9, preferably 6-8. Optionally, the furoic acid can be present in an amount of at least 0.1 mM, at least 0.5 mM, at least 1 mM, at least 5 mM, at least 10 mM, at least 50 mM, at least 0.1M, or at least 0.5M. Optionally, the contacting step can be conducted at a temperature suitable for the HmfF polypeptide to catalyze the reaction, such as a temperature in a range of 35-60 degrees C., including 45-55 degrees C. Optionally, the contacting step may be conducted under pressure, such as greater than 1 bar or greater than atmospheric or ambient pressure to drive the transformation of furoic acid to at least FDCA. Optionally, the pressure can be in greater than 2, 3, 4, 5, or 10 bar.

The natural biological pathway of degradation or breaking down of HMF by certain microorganisms has been studied and reported as involving the successive oxidation reactions going from HMF to 2,5-furandicarboxylic acid (FDCA). As noted above, this is one known method of enzymatically producing FDCA, with the precursor being HMF. Also, it has been reported that the final step in the biological pathway of HMF degradation involves decarboxylation of FDCA to furoic acid, in which the decarboxylation step has been shown to be dependent on two gene products or enzymes, HmfF and HmfG of Cupriavidus basilensis, which are homologous to the enzymes UbiD and UbiX of Escherichia coli, respectively. The fungal enzyme homologous to the UbiD enzyme is the Fdc1 enzyme. Most UbiD-like enzymes, including Fdc1, act as decarboxylases in vivo.

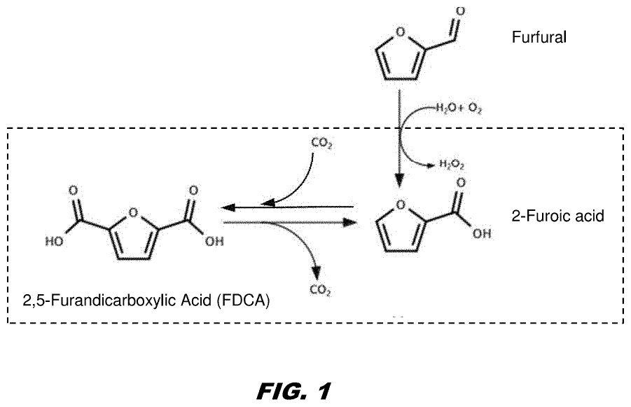

It has now been found, however, certain UbiD-like enzymes can also catalyse a carboxylase reaction from furoic acid to FDCA under certain induced conditions, particularly in the presence of carbon dioxide. FIG. 1 illustrates the reaction scheme of the carboxylation and decarboxylation reactions going from FDCA to furoic acid and vice versa.

While a crystal structure of an HmfF polypeptide in complex with furoic acid is not yet available, the crystal structure of Fdc1 in complex with the substrate of cinnamic acid is available. Using this structure of Fdc1, FDCA was placed into the active site of HmfF in similar position with respect to the prFMN cofactor, which had been identified as assisting in the decarboxylase activity of Fdc1 (see Payne et al. (2015)). This new cofactor supports α,β-unsaturated acid decarboxylation via 1,3-dipolar cycloaddition (Nature 522, 497-501). Positioning places the furan oxygen within hydrogen bonding distance of His297, and locates the distal carboxylate adjacent to Arg305 and/or Arg332. All three residues are conserved amongst the polypeptide comprising the amino acid sequence of SEQ ID NO:1 and SEQ ID NO:2 but not present in other UbiD-like enzymes, such as UbiD and Fdc1, in which these have not been reported to have any activity with furoic acid. Indeed, the sequence identity of UbiD and Fdc1 with a HmfF protein, such as the polypeptide comprising the amino acid sequence of SEQ ID NO:1 or SEQ ID NO:2, is below 34%. Moreover, these non-HmfF-UbiD-like enzymes do not comprise (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2. Table 1 below provides the amino acid sequence identity and amino acid positions for these non-HmfF-UbiD-like enzymes as compared to the amino acid sequence of SEQ ID NO:1 and SEQ ID NO:2.

TABLE 1

| Source | Target | % | | | |
|---|---|---|---|---|---|
| Sequence | Sequence | Identity | H297 | R305 | R332 |
| SEQ ID NO: 1 | A. niger Fdc1 | 21.8% | H | A | T |
| SEQ ID NO: 1 | S. cerevisiae Fdc1 | 19.7% | H | A | L |
| SEQ ID NO: 1 | C. dubliniensis | 21.2% | H | S | L |
| | Fdc1 | | | | |
| SEQ ID NO: 1 | E. coli MG1655 | 26.2% | E | L | Y |
| | UbiD | | | | |
| SEQ ID NO: 2 | A. niger Fdc1 | 20.8% | H | A | T |
| SEQ ID NO: 2 | S. cerevisiae Fdc1 | 19.0% | H | A | L |
| SEQ ID NO: 2 | C. dubliniensis | 19.5% | H | S | L |
| | Fdc1 | | | | |
| SEQ ID NO: 2 | E. coli MG1655 | 23.6% | E | L | Y |
| | UbiD | | | | |

Accordingly, the present disclosure provides a method for preparing a 2,5-furandicarboxylic acid (“FDCA”) comprising: contacting a polypeptide comprising an amino acid sequence that has greater than 34%, optionally at least 40%, optionally at least 50%, optionally at least 60%, optionally at least 70%, optionally at least 80%, optionally at least 90%, or optionally at least 91%, 92%, 93%, 94%, 95%, 96%, 97%, 98%, or 99% sequence identity with an amino acid sequence set out in SEQ ID NO:1 or SEQ ID NO:2 with a furoic acid in the presence of carbon dioxide, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

The polynucleotide sequence of SEQ ID NO: 3 or 4 encodes a polypeptide having the amino acid sequence of SEQ ID NO:1 or SEQ ID NO:2, respectively. An HmfF polypeptide comprising an amino acid sequence of SEQ ID NO:1 or SEQ ID NO:2 may be encoded by a polynucleotide sequence set out in SEQ ID NO: 3 or 4, respectively. One of ordinary skill would know how to generate a polynucleotide sequence for an HmfF polypeptide as described herein. Based on the amino acid sequences provided in SEQ ID NOs:1 and SEQ ID NO:2 and/or the nucleotide sequences provided in SEQ ID NOs: 3 and 4, the skilled person will be able to construct suitable probes and/or primers to isolate a nucleotide sequence coding for a HmfF polypeptide as described herein.

Alternatively, based on the amino acid sequences provided in SEQ ID NOs:1 and SEQ ID NO:2 and/or the polynucleotide sequences provided in SEQ ID NOs: 3 and 4, the skilled person may obtain synthesized sequences coding for an HmfF polypeptide from commercial sources, as gene synthesis is becoming increasingly available. Synthetic sequences may be purchased for example from Geneart A.G. (Regensburg, Germany) or from Genscript USA Inc. (Piscataway, N.J., USA) to name but a few.

An HmfF polypeptide as described herein includes variants of a polypeptide comprising the amino acid sequence of SEQ ID NO:1 or SEQ ID NO:2. As used herein, a “variant” with respect to a polypeptide means a polypeptide in which the amino acid sequence differs from the base sequence from which it is derived in that one or more amino acids within the sequence are substituted for other amino acids. For example, a variant of SEQ ID NO:1 or SEQ ID NO:2 has similar carboxylase and decarboxylase activity as SEQ ID NO:1 or SEQ ID NO:2. It may have an amino acid sequence at least about 34%identical to SEQ ID NO:1 or SEQ ID NO:2, for example, at least about 45%, at least 50%, at least 55%, at least 60%, at least 65%, at least 70%, at least 80%, at least 85%, at least 90%, or at least 95% identical SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

Table 2 lists additional potentially suitable variants, which have at least 34% sequence identity with SEQ ID NO:1 and the conserved and/or exemplary functional substitution at H297 and R305 or R332, with reference to GenBank accession numbers.

TABLE 2

| | | % Identity | | | |
|---|---|---|---|---|---|
| SEQ | | with SEQ | | | |
| ID NO: | Description of sequence | ID NO: 1 | H297 | R305 | R332 |
| 1 | Pelotomaculum | 100% | H | R | R |
| | thermopropionicum | | | | |
| | HmfF polypeptide | | | | |
| | [WP_012031668.1] | | | | |
| 2 | Geobacillus kaustophilus | 55% | H | R | R |
| | HmfF polypeptide | | | | |
| 5 | Desulfotomaculum nigrificans | 65.70% | H | R | R |
| | UbiD family decarboxylase | | | | |
| | [WP_003542232.1] | | | | |
| 6 | Desulfotomaculum nigrificans | 65.50% | H | R | R |
| | UbiD family decarboxylase | | | | |
| | [WP_013810443.1] | | | | |
| 7 | Desulfotomaculum | 65.00% | H | R | R |
| | thermosubterraneum | | | | |
| | UbiD family decarboxylase | | | | |
| | [WP_072868330.1] | | | | |
| 8 | Desulfotomaculum | 65.40% | H | R | R |
| | thermosubterraneum DSM | | | | |
| | 16057 2,5-furandicarboxylate | | | | |
| | decarboxylase | | | | |
| | 1 [SHI93859.1] | | | | |
| 9 | Desulfotomaculum kuznetsovii | 64.80% | H | R | R |
| | DSM 6115 UbiD | | | | |
| | family decarboxylase | | | | |
| | [AEG14240.1] | | | | |
| 10 | Calderihabitans maritimus | 67.30% | H | R | R |
| | UbiD family decarboxylase | | | | |
| | [WP_088552629.1] | | | | |
| 11 | Moorella mulderi UbiD | 65.00% | H | R | R |
| | family decarboxylase | | | | |
| | [WP_062283431.1] | | | | |
| 12 | Desulfurispora thermophila | 64.50% | H | R | R |
| | UbiD family decarboxylase | | | | |
| | [WP_018085023.1] | | | | |
| 13 | Moorella thermoacetica | 64.20% | H | R | R |
| | UbiD family decarboxylase | | | | |
| | [WP_069588023.1] | | | | |
| 14 | Moorella glycerini | 64.40% | H | R | R |
| | UbiD family decarboxylase | | | | |
| | [WP_054935752.1] | | | | |
| 15 | Peptococcaceae bacterium | 61.90% | H | R | R |
| | BRH_c23 | | | | |
| | 3-octaprenyl-4-hydroxybenzoate | | | | |
| | carboxy-lyase [KJS48380.1] | | | | |
| 16 | Fictibacillus enclensis | 53.60% | H | R | R |
| | UbiD family decarboxylase | | | | |
| | [WP_061973934.1] | | | | |
| 17 | Desulfotomaculum putei | 58.10% | H | R | R |
| | UbiD family decarboxylase | | | | |
| | [WP_073240124.1] | | | | |
| 18 | Oscillibacter sp. PC13 | 57.80% | H | R | R |
| | UbiD family decarboxylase | | | | |
| | [WP_091127730.1] | | | | |
| 19 | Desulfotomaculum putei | 58.30% | H | R | R |
| | DSM 12395 | | | | |
| | 2,5-furandicarboxylate | | | | |
| | decarboxylase | | | | |
| | 1 [SHF55034.1] | | | | |
| 20 | Sporomusa acidovorans | 61.60% | H | R | R |
| | UbiD family decarboxylase | | | | |
| | [WP_093793511.1] | | | | |
| 21 | Fictibacillus solisalsi | 53.60% | H | R | R |
| | UbiD family decarboxylase | | | | |
| | [WP_090236331.1] | | | | |
| 22 | Desulfovibrio | 58.00% | H | R | R |
| | dechloracetivorans UbiD | | | | |
| | family decarboxylase | | | | |
| | [WP_071545948.1] | | | | |
| 23 | Thermoflavimicrobium | 56.10% | H | R | R |
| | dichotomicum UbiD family | | | | |
| | decarboxylase | | | | |
| | [WP_093231483.1] | | | | |
| 24 | Oscillibacter sp. 1-3 UbiD | 58.90% | H | R | R |
| | family decarboxylase | | | | |
| | [WP_016322520.1] | | | | |
| 25 | Bacillus sp. FJAT-20673 UbiD | 53.00% | H | R | R |
| | family decarboxylase | | | | |
| | [WP_063575933.1] | | | | |
| 26 | Bacillus simplex 3-octaprenyl-4- | 52.80% | H | R | R |
| | hydroxybenzoate | | | | |
| | carboxy-lyase | | | | |
| | [WP_095395888.1] | | | | |
| 27 | Dethiosulfatibacter | 56.80% | H | R | R |
| | aminovorans UbiD | | | | |
| | family decarboxylase | | | | |
| | [WP_073046793.1] | | | | |
| 28 | Aeribacillus pallidus | 55.20% | H | R | R |
| | UbiD family decarboxylase | | | | |
| | [WP_044898855.1] | | | | |
| 29 | Brevibacillus sp. WF146 | 55.20% | H | R | R |
| | UbiD family decarboxylase | | | | |
| | [WP_065067623.1] | | | | |
| 30 | Brevibacillus thermoruber | 55.20% | H | R | R |
| | UbiD family decarboxylase | | | | |
| | [WP_051188095.1] | | | | |
| 31 | Rhodoplanes sp. Z2-YC6860 | 46.50% | H | K | R |
| | UbiD family decarboxylase | | | | |
| | [WP_068031807.1] | | | | |
| 32 | Rhodoplanes sp. Z2-YC6860 | 46.10% | H | K | R |
| | UbiD family decarboxylase | | | | |
| | [AMN43067.1] | | | | |
| 34 | Acidobacteria bacterium | 34.00% | H | R | R |
| | RIFCSPLOW- | | | | |
| | O2_02_FULL_68_18 | | | | |
| | 4-hydroxybenzoate | | | | |
| | decarboxylase | | | | |
| | [OFW00262.1] | | | | |

Table 3 lists potentially suitable variants, which have at least 34% sequence identity with SEQ ID NO: 2 and the conserved and/or exemplary functional substitution at H297 and R305 or R332, with reference to GenBank accession numbers.

TABLE 3

| | | % Identity | | | |
|---|---|---|---|---|---|
| SEQ | | with Seq | | | |
| ID NO | Description of sequence | No. 2 | H297 | R305 | R332 |
| 1 | Pelotomaculum | 55% | H | R | R |
| | thermopropionicum | | | | |
| | HmfF polypeptide | | | | |
| 2 | Geobacillus kaustophilus | 100% | H | R | R |
| | HmfF polypeptide | | | | |
| | [WP_011229502.1] | | | | |
| 23 | Thermoflavimicrobium | 80.90% | H | R | R |
| | dichotomicum | | | | |
| | UbiD family decarboxylase | | | | |
| | [WP_093231483.1] | | | | |
| 26 | Bacillus simplex 3-octaprenyl-4- | 77.50% | H | R | R |
| | hydroxybenzoate carboxy-lyase | | | | |
| | [WP_095395888.1] | | | | |
| 25 | Bacillus sp. FJAT-20673 | 77.50% | H | R | R |
| | UbiD family decarboxylase | | | | |
| | [WP_063575933.1] | | | | |
| 38 | Effusibacillus lacus | 79.70% | H | R | R |
| | 3-octaprenyl-4- | | | | |
| | hydroxybenzoate carboxy-lyase | | | | |
| | [WP_096181721.1] | | | | |
| 39 | Aneurinibacillus | 79.20% | H | R | R |
| | terranovensis | | | | |
| | UbiD family decarboxylase | | | | |
| | [WP_035100811.1] | | | | |
| 40 | Paenibacillus naphthalenovorans | 77.20% | H | R | R |
| | UbiD family decarboxylase | | | | |
| | [WP_062409774.1] | | | | |
| 41 | Domibacillus tundrae UbiD | 76.90% | H | R | R |
| | family decarboxylase | | | | |
| | [WP_046179493.1] | | | | |
| 42 | Domibacillus iocasae UbiD | 76.70% | H | R | R |
| | family decarboxylase | | | | |
| | [WP_069939921.1] | | | | |
| 43 | Paenibacillus naphthalenovorans | 76.80% | H | R | R |
| | UbiD family decarboxylase | | | | |
| | [WP_074728556.1] | | | | |
| 44 | Brevibacillus thermoruber UbiD | 76.50% | H | R | R |
| | family decarboxylase | | | | |
| | [WP_051967874.1] | | | | |
| 30 | Brevibacillus thermoruber UbiD | 76.30% | H | R | R |
| | family decarboxylase | | | | |
| | [WP_051188095.1] | | | | |
| 45 | Brevibacillus sp. OK042 UbiD | 80.00% | H | R | R |
| | family decarboxylase | | | | |
| | [WP_092277161.1] | | | | |
| 28 | Aeribacillus pallidus UbiD | 76.30% | H | R | R |
| | family decarboxylase | | | | |
| | [WP_044898855.1] | | | | |
| 29 | Brevibacillus sp. WF146 UbiD | 76.10% | H | R | R |
| | family decarboxylase | | | | |
| | [WP_065067623.1] | | | | |
| 46 | Bacillus sp. OV194 UbiD | 74.40% | H | R | R |
| | family decarboxylase | | | | |
| | [WP_091005856.1] | | | | |
| 47 | Fictibacillus sp. FJAT-27399 | 74.40% | H | R | R |
| | UbiD family decarboxylase | | | | |
| | [WP_062232290.1] | | | | |
| 48 | Bacillus sp. FJAT-26652 | 74.20% | H | R | R |
| | UbiD family decarboxylase | | | | |
| | [WP_053354189.1] | | | | |
| 16 | Fictibacillus enclensis | 72.60% | H | R | R |
| | UbiD family decarboxylase | | | | |
| | [WP_061973934.1] | | | | |
| 49 | Bacillus kribbensis | 76.10% | H | R | R |
| | UbiD family decarboxylase | | | | |
| | [WP_035321686.1] | | | | |
| 21 | Fictibacillus solisalsi | 72.40% | H | R | R |
| | UbiD family decarboxylase | | | | |
| | [WP_090236331.1] | | | | |
| 50 | Bacillus xerothermodurans | 75.60% | H | R | R |
| | UbiD family decarboxylase | | | | |
| | [WP_089200121.1] | | | | |
| 51 | Brevibacillus panacihumi | 76.20% | H | R | R |
| | UbiD family decarboxylase | | | | |
| | [WP_023558113.1] | | | | |
| 52 | Alicyclobacillus vulcanalis | 55.00% | H | R | R |
| | UbiD family decarboxylase | | | | |
| | [WP_084182558.1] | | | | |
| 53 | Alicyclobacillus acidocaldarius | 60.00% | H | R | R |
| | UbiD family decarboxylase | | | | |
| | [WP_014463641.1] | | | | |
| 54 | Alicyclobacillus vulcanalis 2,5- | 64.60% | H | R | R |
| | furandicarboxylate decarboxylase | | | | |
| | 1 [SIS94084.1] | | | | |
| 55 | Alicyclobacillus mali | 60.90% | H | R | R |
| | UbiD family decarboxylase | | | | |
| | [WP_067848851.1] | | | | |
| 56 | Bacillus ligniniphilus | 60.30% | H | R | R |
| | UbiD family decarboxylase | | | | |
| | [WP_017729030.1] | | | | |
| 57 | Jeotgalibacillus soli | 61.30% | H | R | R |
| | Cunha et al. 2012 | | | | |
| | UbiD family decarboxylase | | | | |
| | [WP_041089354.1] | | | | |
| 5 | Desulfotomaculum nigrificans | 58.60% | H | R | R |
| | UbiD family decarboxylase | | | | |
| | [WP_003542232.1] | | | | |
| 6 | Desulfotomaculum nigrificans | 58.40% | H | R | R |
| | UbiD family decarboxylase | | | | |
| | [WP_013810443.1] | | | | |
| 17 | Desulfotomaculum putei | 56.50% | H | R | R |
| | UbiD family decarboxylase | | | | |
| | [WP_073240124.1] | | | | |
| 31 | Rhodoplanes sp. Z2-YC6860 | 49.60% | H | K | R |
| | UbiD family decarboxylase | | | | |
| | [WP_068031807.1] | | | | |
| 32 | Rhodoplanes sp. Z2-YC6860 | 49.10% | H | K | R |
| | UbiD family decarboxylase | | | | |
| | [AMN43067.1] | | | | |
| 61 | Caloramator mitchellensis | 44.40% | H | R | R |
| | UbiD family decarboxylase | | | | |
| | [WP_057976570.1] | | | | |

The variants are functional variants in that the variant sequence has enzymatic activity characteristics as compared to the enzyme having the non-variant amino acid sequence specified herein (and this is the meaning of the term “functional variant” as used throughout this specification) and are able to catalyze the FDCA in contact with furoic acid and in the presence of carbon dioxide. Preferably, the functional variants have similar or higher carboxylase and decarboxylase activity that that of the enzyme having an amino acid of SEQ ID NO:1 or SEQ ID NO:2. The enzymatic activity of variants may be assessed, for example, by comparing the rate of interconversion between furoic acid and CO₂ and FDCA by a variant to the rate achieved by a polypeptide comprising an amino acid sequence of SEQ ID NO:1 or SEQ ID NO:2 under the same conditions. For a functional variant, this rate may be the same or similar, for example at least about 30%, 40%, 50%, 60%, 70%, 80%, 90%, 95%, or greater than the rate achieved by a polypeptide comprising an amino acid sequence of SEQ ID NO:1 or SEQ ID NO:2 under the same conditions.

As noted above, amino acid substitutions in variants may be regarded as “conservative” where an amino acid is replaced with a different amino acid with broadly similar properties. Non-conservative substitutions are where amino acids are replaced with amino acids of a different type.

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

Functional substitutions that are non-conservative substitutions are possible also, provided that these do not interrupt the enzyme activities of the polypeptides, as defined elsewhere herein.

Broadly speaking, fewer non-conservative substitutions than conservative substitutions will be possible without altering the biological activity of the polypeptides. Determination of the effect of any substitution (and, indeed, of any amino acid deletion or insertion) is wholly within the routine capabilities of the skilled person, who can readily determine whether a variant polypeptide retains the carboxylase and decarboxylase enzyme activity as described herein. For example, when determining whether a variant of the polypeptide falls within the scope of the invention (i.e., is a “functional variant or fragment” as defined above), the skilled person will determine whether the variant or fragment retains the substrate converting enzyme activity which is at least about 60%, preferably at least about 70%, more preferably at least about 80%, yet more preferably about 90%, about 95%, about 96%, about 97%, about 98%, about 99% or about 100% the activity of the non-variant polypeptide. In some cases, the variant may have enzyme activity which is greater than 100% the activity of the non-variant polypeptide, i.e., the variant may have improved enzyme activity compared to the non-variant and increase the rate of conversion of the substrate relevant to the particular enzyme compared to the rate achieved by the non-variant under the same conditions (e.g., substrate concentration, temperature). All such variants are within the scope of the aspects described herein.

The present disclosure also provides embodiments that encompass variant nucleic acid sequences encoding the polypeptides of the invention. The term “variant” in relation to a nucleic acid sequence means any substitution of, variation of, modification of, replacement of, deletion of, or addition of one or more nucleic acid(s) from or to a polynucleotide sequence, providing the resultant polypeptide sequence encoded by the polynucleotide exhibits at least the same or similar enzymatic properties as the polypeptide encoded by the basic sequence. The term therefore includes allelic variants and also includes a polynucleotide (a “probe sequence”) which substantially hybridizes to the polynucleotide sequence of the present invention. Such hybridisation may occur at or between low and high stringency conditions. In general terms, low stringency conditions can be defined as hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tₘ of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (10).

Using the standard genetic code, further nucleic acid sequences encoding the polypeptides may readily be conceived and manufactured by the skilled person, in addition to those disclosed herein. The nucleic acid sequence may be DNA or RNA and, where it is a DNA molecule, it may for example comprise a cDNA or genomic DNA. The nucleic acid may be contained within an expression vector, as described elsewhere herein.

In a typical approach, gene libraries can be screened to isolate alternative polynucleotides which are suitable. The libraries may be constructed from microorganisms from the superkingdom of Bacteria. These microorganisms may belong to the phylum of Proteobacteria, more specifically to the class of Alphaproteobacteria or Betaproteobacteria. The Alphaproteobacteria may belong to the order of Rhizobiales, to the families of Bradyrhizobiaceae or Methylobacteriaceae. The Bradyrhizobiaceae may belong to the genus of Bradyrhizobium, e.g., Bradyrhizobium japonicum, or to the genus of Afipia. The Methylobacteriaceae may belong to the genus of Methylobacterium, e.g., Methylobacterium nodularis or Methylobacterium radiotolerans. The Betaproteobacteria may belong to the order of Burkholderiales, more specifically the family of Burkholderiaceae. They may belong to the genus Cupriavidus, e.g., Cupriavidus basilensis; or to the genus Ralstonia, e.g., Ralstonia eutropha; or to the genus Burkholderia, e.g., Burkholderia phymatum, Burkholderia phytofirmans, Burkholderia xenovorans, or Burkholderia graminis. The bacteria may also belong to the phylum of Firmicutes, more specifically the class of Bacilli, more specifically the order of Bacillales. The Bacillales may belong to the family of Bacillaceae, more specifically to the genus Geobacillus, e.g., Geobacillus kaustophilus. Alternatively, the microorganisms may belong to the superkingdom of Archaea, more specifically the phylum of Euryarchaeota, or the phylum of Crenarchaeota. The Euryarchaeota may belong to an unclassified genus, e.g., Cand. Parvarchaeum acidiphilum, or to the class of Thermoplasmata, more specifically the order of Thermoplasmatales. The Thermoplasmatales may belong to the family of Thermoplasmataceae, more specifically the genus Thermoplasma, e.g., Thermoplasma acidophilum or Thermoplasma volcanium. The Crenarchaeota may belong to the class of Thermoprotei, more specifically the order of Sulfolobales. The Sulfolobales may belong to the family of Sulfolobaceae, more specifically the genus Sulfolobus, e.g., Sulfolobus acidocaldarius, Sulfolobus islandicus, Sulfolobus solfataricus, or Sulfolobus tokodaii; or to the genus of Metallosphaera, e.g., Metallosphaera sedula. The Thermoprotei may also belong to the order of Thermoproteales, family of Thermoproteaceae. The Thermoproteaceae may belong to the genus Vulcanisaeta, e.g., Vulcanisaeta distribute, or to the genus Caldivirga, e.g., Caldivirga maquilingensis.

The present disclosure also relates to vectors, including cloning and expression vectors, comprising the polynucleotide or a functional equivalent thereof that encodes for an HmfF protein and methods of growing, transforming or transfecting such vectors in a suitable host cell, for example under conditions in which expression of an HmfF protein occurs.

A polynucleotide encoding for an HmfF protein can be incorporated into a recombinant replicable vector, for example a cloning or expression vector. As such, the present disclosure also provides for a vector or plasmid or isolated and purified polynucleotide comprising a nucleic acid sequence encoding a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

The vector may be used to replicate the nucleic acid in a compatible host cell. Thus, the present disclosure also provides a method of making an HmfF polynucleotide by introducing a polynucleotide encoding for an HmfF polypeptide into a replicable vector, introducing the vector into a compatible host cell, and growing the host cell under conditions which bring about replication of the vector. The vector may be recovered from the host cell. Suitable host cells are described below.

The vector into which the expression cassette or polynucleotide of the invention is inserted may be any vector which may conveniently be subjected to recombinant DNA procedures, and the choice of the vector will often depend on the host cell into which it is to be introduced.

A vector according to the invention may be an autonomously replicating vector, i.e. a vector which exists as an extra-chromosomal entity, the replication of which is independent of chromosomal replication, e.g., a plasmid. Alternatively, the vector may be one which, when introduced into a host cell, is integrated into the host cell genome and replicated together with the chromosome (s) into which it has been integrated.

As mentioned above, one type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as cosmid, viral vectors (e.g., replication defective retroviruses, adenoviruses and adeno-associated viruses), phage vectors and transposons and plasposons, which serve equivalent functions.

The skilled person will be able to construct the vectors described herein based on the amino acid and polynucleotide sequences provided, his/her knowledge of the art and commercially available means.

Accordingly, the host cell may be genetically modified by any manner known to be suitable for this purpose by the person skilled in the art. This includes the introduction of the gene of interest encoding an HmfF polypeptide on a plasmid or other expression vector which reproduces within the host cell. Alternatively, the plasmid or part of the plasmid or may integrate into the host genome, for example by homologous recombination. To carry out genetic modification, DNA can be introduced or transformed into cells by natural uptake or mediated by processes such as electroporation or conjugation. Genetic modification can involve expression of a gene under control of an introduced promoter. The introduced DNA may encode a protein which could act as an enzyme or could regulate the expression of further genes.

Such a host cell may comprise a nucleic acid sequence encoding an HmfF polypeptide. Accordingly, the present disclosure also provides for a recombinant host cell comprising a nucleic acid sequence encoding a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

In another embodiment, an HmfF polypeptide or functional variant or fragment of either of these may be expressed in a non-micro-organism cell such as a cultured mammalian cell or a plant cell or an insect cell. Mammalian cells may include CHO cells, COS cells, VERO cells, BHK cells, HeLa cells, Cvl cells, MDCK cells, 293 cells, 3T3 cells, and/or PC12 cells.

The recombinant host cell or micro-organism may be used to express the enzymes mentioned above for use according to methods described herein, such as in in vivo or in vitro systems (e.g., a cell lysate system known to one of ordinary skill in the art). For instance, a suitable host cell may be modified as described herein to express an HmfF polypeptide of the present disclosure, where such HmfF polypeptide may be released from the host cell in a lysate (as known to one of ordinary skill), or optionally subsequently purified, using methods known to one of ordinary skill and used outside of such host cell in an in vitro process.

As such, the present disclosure provides for a cell-free lysate composition comprising:
f. a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2;
g. furoic acid; and
h. carbon dioxide.

For example, after a host cell has expressed and contains the HmfF polypeptide, it may be opened using methods known to one of ordinary skill to release the HmfF polypeptide where such HmfF polypeptide may catalyse a reaction outside of the host cell. The lysate or solution containing the opened host cell and released HmfF polypeptide may be considered “cell-free” even though the lysate or solution may still contain remnants of the opened host cell because in vitro reactions can take place in such lysate. Once released or freed from the host cell and in the lysate, the HmfF polypeptide may be combined with furoic acid and carbon dioxide to carry out the production of FDCA in vitro. Optionally, the HmfF polypeptide released from the host cell may be purified or partially purified using methods known to one of ordinary skill in the art so it may be added exogenously.

The cell lysate system may be preferably conducted in a reactor that is under pressure, such as greater than 1 bar or greater than atmospheric or ambient pressure to drive the transformation of furoic acid to at least FDCA. For instance, the pressure can be in a range of 1 bar to 10 bar, preferably 5 bar to 10 bar. Optionally, the cell lysate system can involve a multiphase reaction system to deliver carbon dioxide to the liquid phase comprising furoic acid and an HmfF polypeptide, by at least diffusing a gas composition comprising carbon dioxide into the liquid phase.

The present disclosure provides an option to produce FDCA from furoic acid via an enzymatic route at various volumes, including bench-scale to large-scale. For instance, such a method can comprise: providing a reactor comprising furoic acid, carbon dioxide, a HmfF polypeptide comprising an amino acid sequence that has greater than 34% sequence identity with an amino acid sequence set out in SEQ ID NO:1 or SEQ ID NO:2; and furoic acid, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2, and allowing the HmfF polypeptide, furoic acid, and carbon dioxide to remain in the reactor for a period of time to allow production of FDCA. Optionally, the carbon dioxide can be present in the reactor in an amount greater than ambient conditions, such as at least 400 ppm, at least 600 ppm, at least 800 ppm, or at least 1000 ppm. Optionally, the pH in the reactor can be in a range of 5-9, preferably 6-8. Optionally, the furoic acid can be present in an amount of at least 0.1 mM, at least 0.5 mM, at least 1 mM, at least 5 mM, at least 10 mM, at least 50 mM, at least 0.1M, or at least 0.5M. Optionally, the reactor can have a temperature suitable for the HmfF polypeptide to catalyze the reaction, such as a temperature in a range of 35-60 degrees C., including 45-55 degrees C. Optionally, the reactor may be under pressure, such as greater than 1 bar or greater than atmospheric or ambient pressure to drive the transformation of furoic acid to at least FDCA. Optionally, the pressure can be in greater than 2, 3, 4, 5, or 10 bar.

Optionally, the reactor can comprise a gas phase comprising carbon dioxide and an aqueous phase comprising furoic acid and the HmfF polypeptide. The carbon dioxide may be provided to the reactor as a gas or vapor. Optionally, at least a portion of the carbon dioxide may be under supercritical conditions. For example, it has been demonstrated that supercritical carbon dioxide can be used with UbiD-like enzymes to drive reactions. Dibenedetto et al, First in vitro use of the phenylphosphate carboxylase enzyme in supercritical CO2 for the selective carboxylation of phenol to 4-hydroxybenzoic acid. Environ. Chem Lett. 3, 145-148 (2006) and Aresta M., Dibenedetto A., Quaranta E. (2016) Enzymatic Conversion of CO2 (Carboxylation Reactions and Reduction to Energy-Rich Cl Molecules). In: Reaction Mechanisms in Carbon Dioxide Conversion. Springer, Berlin, Heidelberg.

The method may be carried out in a semi-continuous or continuous manner, such as a continuous stirred tank reactor (CSTR), or in a batch reactor, where the HmfF polypeptide in either instance is available in the aqueous phase. In circumstances where the HmfF polypeptide is in the liquid phase, the carbon dioxide may be injected into the reactor through a sparger, optionally a microbubble sparger, to maximize the surface area of bubbles that contain carbon dioxide in gas phase to facilitate contact of the carbon dioxide with the HmfF polypeptide and furoic acid in the liquid phase. Optionally, if a CSTR is used, then an agitation speed and/or strength may be as high as possible without damaging enzymes. For instance, impellers or other similar mechanical mixing means usually work well for bubble distribution. If the liquid phase comprises an aqueous phase and an organic phase, suitable impellers may be selected such as hydrofoils. Preferably, the reactor optimally is taller than wider (L/D 3 to 1) to give some height for the bubbles to rise and diffuse into solution.

Additionally or alternatively, the HmfF polypeptide may be immobilized in a solid phase and the carbon dioxide and furoic acid are in liquid phase using means known to one of ordinary skill such as a “pre-saturation” vessel under pressure to place at least a portion of the carbon dioxide into liquid phase, which can then be flown through a fixed bed support containing the enzyme.

The FDCA may be recovered using methods known to one of ordinary skills, such as precipitating the FDCA through lowering the temperature of the liquid phase or other physical or chemical known methods.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### EXAMPLES

Cloning of Pelotomaculum thermopropionicum (SEQ ID NO:1), Geobacillus kaustophilus (SEQ ID NO:2), which are two examples of an HmfF enzyme as described herein, for E. coli heterologous expression.

The Pelotomaculum thermopropionicum 2,5-furandicarboxylic acid decarboxylase (HmfF) gene (WP_01203668), Brevibacillus thermoruber HmfF (WP_051188095), Desulfotomaculum geothermicum HmfF (SFR07794) and G. kaustophilus HmfF gene (WP_011229502) were codon optimized to remove codons that were rare in E. coli and synthesised (Genscript). The HmfF genes were amplified using Phusion polymerase (NEB) and the PCR product was cloned into the NdeI and XhoI sites of the respective pET expression plasmid (MerckMillipore) using Infusion HD (Clontech) and transformed into E. coli NEB5A.

To produce N-terminally tagged proteins the P. thermopropionicum HmfF PCR was performed using primers Ptherm28aF (CGCGCGGCAGCCATATGTCCCACTCCCTGCG) and Ptherm28a/21bR (GGTGGTGGTGCTCGAGTTATTATTCCAGGTAGTCTGCCAG), the B. thermoruber HmfF PCR was performed using Bv28a Ntag F (CGCGGCAGCCATATGCGTGCCAAAACC) and Bv28a/30a Stop R (GTGGTGGTGCTCGAGTTACAGATAATCTTCCAGACG) and the Desulfotomaculum geothermicum HmfF PCR performed using DgeoFDCAopt_28aF (CGCGCGGCAGCCATATGGCATATAGCCTGCGTG) and DgeoFDCAopt_21b/28aR (GGTGGTGGTGCTCGAGTTATTAATCCAGATAATCATCCAGTTTAATG). The PCR amplified genes were cloned in to pET28a linearized with NdeI and XhoI.

To produce C-terminally tagged proteins the P. thermopropionicum HmfF PCR was performed using primers Ptherm30a/21bF (AAGGAGATATACATATGTCCCACTCCCTGCG) and Ptherm30aR (GGTGGTGGTGCTCGAGTTCCAGGTAGTCTGCCAG) (Eurofins), the B. thermoruber HmfF PCR was performed using Bv30a Ctag F (GGAGATATACATATGCGTGCCAAAACC) and Bv30a Ctag R (GTGGTGGTGCTCGAGCAGATAATCTTCCAGACGAATTTC) and the Desulfotomaculum geothermicum HmfF PCR performed using (AAGGAGATATACATATGGCATATAGCCTGCGTG) and (GGTGGTGGTGCTCGAGATCCAGATAATCATCCAGTTTAATG). The PCR amplified genes were cloned in to pET30a linearized with NdeI and XhoI.

To produce untagged P. thermopropionicum HmfF the gene was amplified using the primers Ptherm30a/21bF (AAGGAGATATACATATGTCCCACTCCCTGCG) and Ptherm28a/21bR (GGTGGTGGTGCTCGAGTTATTATTCCAGGTAGTCTGCCAG) and cloned into pET21b.

Once the sequence of the desired insert was confirmed, the corresponding purified plasmid was transformed into E. coli BL21(DE3). The pET28a and pET30a constructs were co-transformed with ubiXpET21b to provide sufficient levels of prFMN in vivo.

Expression After Cloning

The various HmfF enzymes were expressed in BL21(DE3) grown at 37° C./180 rpm in LB broth supplemented with 50 mg/ml kanamycin and 50 mg/ml ampicillin except in the case of P. thermopropionicum HmfF pET21b where 50 mg/ml streptomycin and 50 mg/ml ampicillin were used. At mid-log phase cells were induced with 0.25 mM IPTG and supplemented with 1 mM MnCl₂, grown overnight at 15° C./180 rpm and then harvested by centrifugation (4° C., 7000 g for 10 minutes).

Purification of His-Tagged HmfF Proteins

Cell pellets were resuspended in buffer A (200 mM KCl, 1 mM MnCl₂, 50 mM Tris pH 7.5) supplemented with DNase, RNase, lysozyme (Sigma) and Complete EDTA-free protease inhibitor cocktail (Roche). Cells were lysed using a French press at 20,000 psi and the lysate was clarified by centrifugation at 125,000 g for 90 minutes. The supernatant was applied to a Ni-NTA agarose column (Qiagen). The column washed with 3 column volumes of buffer A supplemented with 10 mM imidazole and protein eluted in 1 ml fractions with buffer A supplemented with 250 mM imidazole. Samples were subjected to SDS-PAGE analysis and fractions found to contain the purified protein were pooled. Imidazole was removed using a 10-DG desalting column (Bio-Rad) equilibrated with buffer A. Protein was aliquoted and flash frozen until required.

Purification of Untagged P. thermopropionicum HmfF

Cell pellets were resuspended in buffer A (200 mM KCl, 1 mM MnCl₂, 50 mM Tris pH 7.5) supplemented with DNase, RNase, lysozyme (Sigma) and Complete EDTA-free protease inhibitor cocktail (Roche). Cells were lysed using a French press at 20,000 psi and the lysate incubated at 50° C. for 30 minutes to precipitate host proteins. The lysate was clarified by centrifugation at 125,000 g for 90 minutes. The P. thermopropionicum HmfF was precipitated with 30% saturating ammonium sulphate at 4° C., the supernatant was removed following centrifugation and the pellet solubilized in buffer A and subjected to size exclusion chromatography using a HiPrep S200 column (GE Healthcare) equilibrated with buffer A and SEQ ID NO:2 ml fractions collected. Samples were subjected to SDS-PAGE analysis and fractions found to contain the purified protein were pooled. Protein was aliquoted and flash frozen until required.

UV-Vis Spectroscopy/Protein Quantification

UV-Vis absorbance spectra were recorded with a Cary UV-Vis spectrophotometer. The protein concentration was estimated from the A₂₈₀ absorption peak with extinction coefficients calculated from the primary amino acid sequence using the ProtParam program on the ExPASy proteomics server. P. thermopropionicum HmfF was estimated using e₂₈₀=28420 M⁻¹ cm⁻¹, G. kaustophilus HmfF e₂₈₀=31860 M⁻¹ cm⁻¹, Brevibacillus thermoruber HmfF e₂₈₀=33350 M⁻¹ cm⁻¹ and Desulfotomaculum geothermicum HmfF e₂₈₀=32890 M⁻¹ cm⁻¹.

HmfF Decarboxylation Assays Monitored by UV-vis

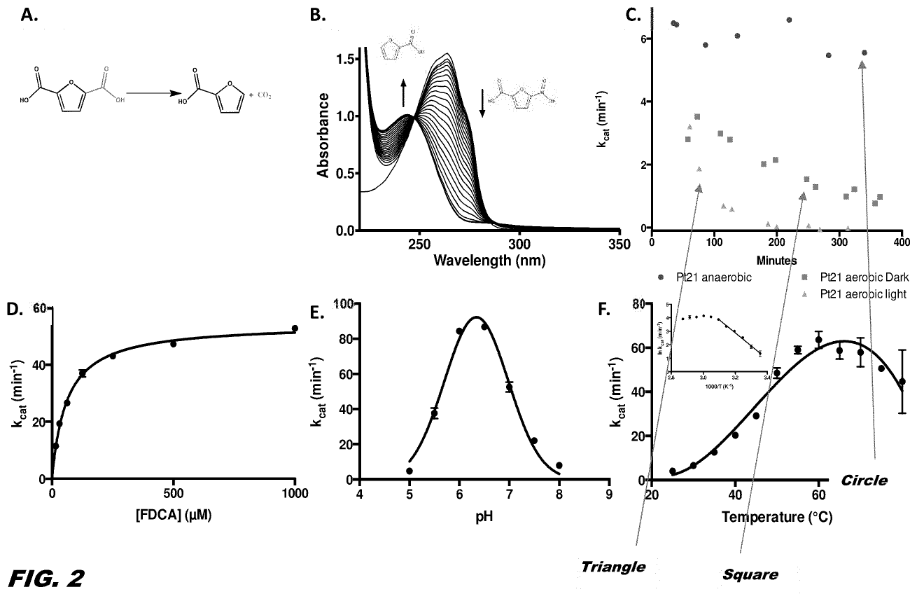

Initial rates of FDCA decarboxylation were determined by monitoring FDCA concentration by UV-vis spectroscopy at 265 nm using an extinction coefficient e₂₆₅=18000 M⁻¹ cm⁻¹, using a Cary 50 Bio spectrophotometer (Varian). Assays were performed against various concentrations of substrate in 350 ml 50 mM KCl, 50 mM NaPi pH 6 in a 1 mm path length cuvette at 50° C. FIG. 2 shows decarboxylation activity assays of a HmfF as described in the present disclosure monitored by UV-vis. In particular, panel A of FIG. 2 shows the decarboxylation reaction. Panel B shows UV-vis observation of the enzymatic conversion of 2,5-furandicarboxylic acid (FDCA) to furoic acid via decarboxylation by P. thermopropionicum HmfF. The initial spectrum of FDCA shows a lₘₐₓ of 265 nm. Over time successive spectra show reduction of the 265 nm peak and appearance of a peak at 245 nm corresponding to furoic acid formation.

Panel C shows that enzyme activity was found to decrease with time (triangle). The rate of inactivation was found to decrease when the protein was stored in the dark (square) or in the absence of oxygen (circle). Panel D shows that steady state kinetic parameters obtained for P. thermopropionicum HmfF with FDCA. Panel E shows the effect of pH on activity, and panel F shows the effect of temperature on activity. Inset, Arrhenius plot of data in (F). Error bars represent SEM, n=3.

HmfF Carboxylation Reactions Assayed by HPLC

Typical assays containing 50 mM furoic acid, 100 mM KPi pH6, 1M KHCO₃ (final pH 7.5), were incubated with and without HmfF enzyme at 50° C. overnight. The sample was centrifuged at 16100 g to remove precipitate and SEQ ID NO:20 μl added to 980 μl 50% v/v H₂O/acetonitrile. Sample analysis was performed using an Agilent 1260 Infinity Series HPLC equipped with a UV detector. The stationary phase was a Kinetex 5 mm C18 100A column, 250×4.6 mm The mobile phase was acetonitrile/water (50/50) with 0.1% TFA at a flow rate of 1 ml/min, and unless otherwise stated detection was performed at a wavelength of 265 nm.

P. thermopropionicum HmfF Catalyses Decarboxylation of FDCA

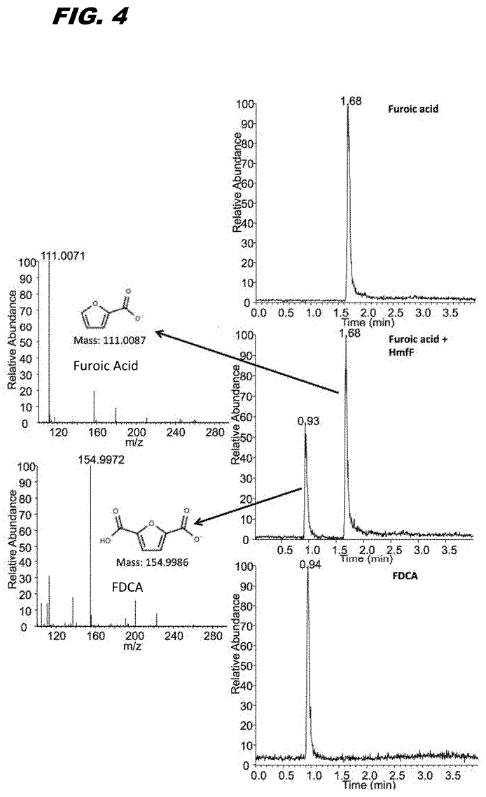

The purified P. thermopropionicum HmfF was capable of decarboxylating 2,5-furandicarboxylic acid to furoic acid, but could not further decarboxylate furoic acid to furan. As can be seen in FIG. 4, FDCA absorbs in the UV region with a lₘₐₓ of 265 nm, allowing the enzyme to be assayed by monitoring changes in 265 nm and hence FDCA depletion. Upon addition of the enzyme to a solution of FDCA, a reduction of the 265 nm peak and appearance of a feature centered at 245 nm occur over time, the latter corresponding to furoic acid formation. HmfF activity was found to rapidly decrease under aerobic conditions. Subsequently, HmfF was purified and assayed under anaerobic conditions. Enzyme activity was found to have a pH optimum between 6 and 6.5, with a temperature maximum of ˜60° C. However, at 55° C. and above the rates appeared to decrease rapidly, indicating enzyme inactivated, making it difficult to obtain accurate initial rates. All subsequent assays were performed at 50° C. An Arrhenius plot of the 25° C.-50° C. data points indicated an activation energy of 80.7 kJ mol⁻¹. At pH 6 and 50° C., the apparent Kₘ and k_{cat} values for FDCA were 61.1 (±3.4) mM and 54.5 (±0.8) min⁻¹ respectively. The P. thermopropionicum HmfF enzyme was found to be highly specific, with no decarboxylation detected for 2,3-furandicarboxylic acid, 5-formyl-2-furoic acid, 5-hydroxymethyl-2-furoic acid, 5-nitro-2-furoic acid, 2,5-thiophenedicarboxylic acid, 2,6-pyridinedicarboxylic acid, terephthalic acid, isophthalic acid or muconic acid.

P. thermopropionicum HmfF and G. kaustophilus HmfF Catalyse Furoic Acid Carboxylation in the Presence of Carbon Dioxide

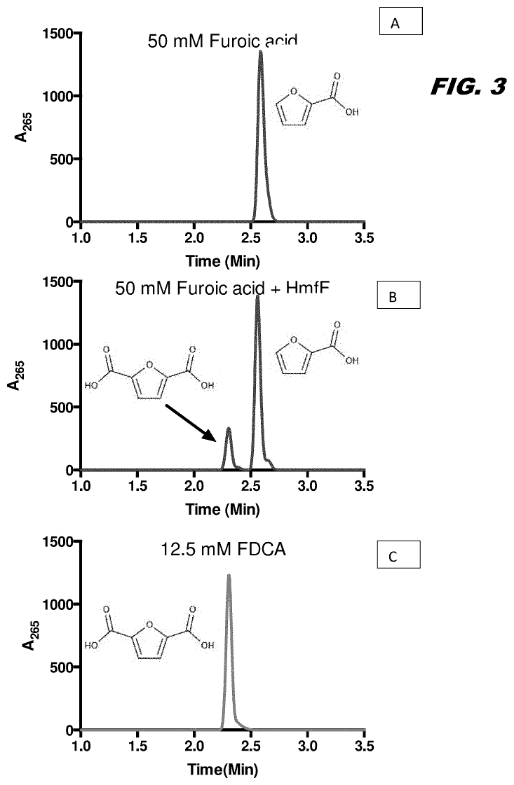

To investigate the ability of HmfF enzymes to catalyse the reverse reaction-carboxylation of furoic acid to produce FDCA, purified P. thermopropionicum HmfF and G. kaustophilus HmfF enzymes were incubated with 50 mM furoic acid and 3 M ammonium bicarbonate at 50° C. overnight. HPLC analysis of the reaction mixtures revealed a peak with retention time of 2.3 minutes that co-migrates with an FDCA standard. Mass spectrometry confirmed that this species had a mass of 154.99 Da, consistent with the expected mass for FDCA. FIG. 3 shows a HPLC chromatogram that demonstrates enzymatic production of FDCA by carboxylation of furoic acid by P. thermopropionicum HmfF. Chromatograms of 12.5 mM FDCA (panel C) and 50 mM furoic acid in 1M KHCO₃ solution incubated in the absence (panel A) and presence (panel B) of the by P. thermopropionicum HmfF enzyme.

P. thermopropionicum HmfF Crystal Structures Reveal FMN Binding Mode

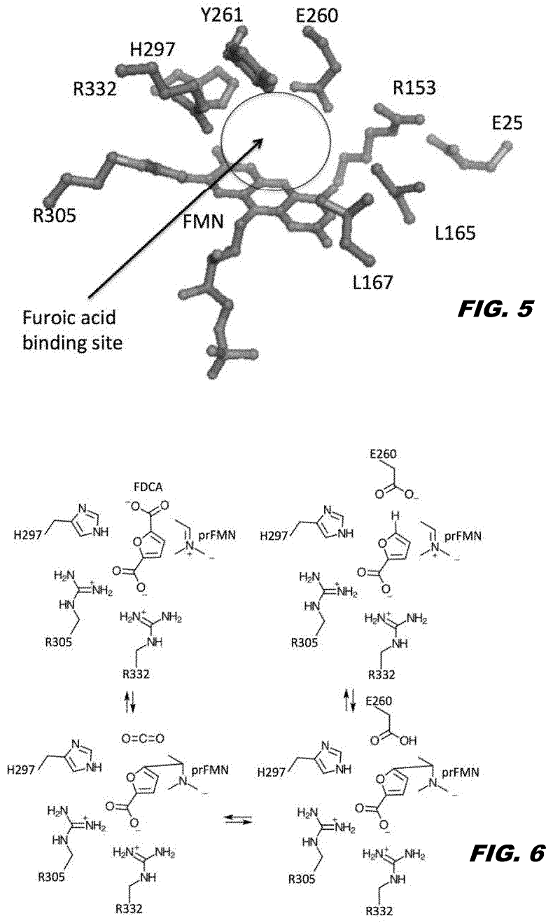

The untagged P. thermopropionicum FDCA decarboxylase was screened against 480 crystallisation conditions. The best crystals belonged to P2₁ spacegroup with a=85 Å, b=139.6 Å, c=136.9 Å, b=93.72° and diffracted to 2.8 Å. The structure revealed six P. thermopropionicum HmfF subunits within the asymmetric unit, forming a hexamer. No electron density corresponding to the cofactor could be detected. FIG. 5 depicts the binding site portion of the P. thermopropionicum HmfF crystal structure in complex with FMN (to 2.7 Å). Key amino acids surrounding the active site are shown in addition to the FMN (flavin mononucleotide), which is an analogue to and can mimic prFMN, where the postulated furoic acid binding site is circled. As can be seen in FIG. 5, upon soaking of the P. thermopropionicum HmfF crystals with FMN in the presence of K⁺ and Mn²⁺, clear electron density was apparent for both the FMN and the associated metal ions (2.7 Å resoution).

P. thermopropionicum HmfF Structure Contains a Putative Furoic Acid Binding Site

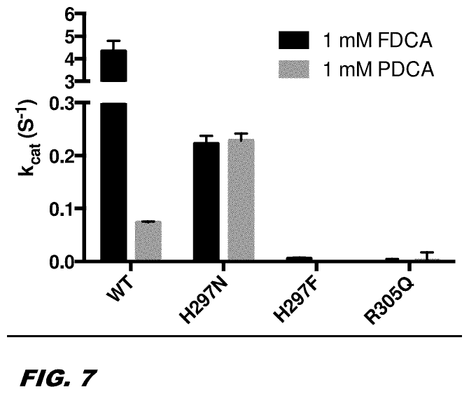

It is believed that all attempts to acquire a crystal structure of the P. thermopropionicum HmfF in complex with substrate, either through soaking or co-crystallisation, have failed. Guided by the structure of Fdc1 in complex with cinnamic acid (5) FDCA was placed into the active site of HmfF in similar position with respect to the prFMN cofactor. This places the furan oxygen within hydrogen bonding distance of His297, and locates the distal carboxylate adjacent to Arg305 and/or Arg332. All three putative substrate binding residues are conserved between P. thermopropionicum HmfF and G. kaustophilus HmfF. FIG. 6 is a schematic representation of structural formula of the P. thermopropionicum HmfF substrate complex and the carboxylation and decarboxylation mechanism. FIG. 6 depicts a plausible mechanism involving binding of furoic acid by H297/R305 and R332 based on the information from the crystal structures. Residues H297, R305 and R332 determine FDCA/furancarboxylic acid binding and orientation.

Mutagenesis of H297 and R305.

Material and Methods for Mutagenesis:

Mutagenesis primers were designed using the QuikChange® Primer Design Program (http://www.genomics.agilent.com/primerDesignProgram.jsp). PCR was performed using Phusion polymerase (NEB). Template was removed by DpnI (NEB) digest and the PCR product transformed into E. coli NEB5-alpha. Once the presence of the desired mutation was confirmed by DNA sequencing, the plasmid was transformed into E. coli BL21(DE3). The three variants transformed into E. coli BL21(DE3) includes one with a mutation of H297N, a second with H297F, and a third of R305Q.

Mutant Expression/Purification.

Protein was expressed in BL21(DE3) grown at 37° C./180 rpm in LB broth supplemented with 50 μg/ml ampicillin At mid-log phase cells were induced with 0.25 mM IPTG and grown overnight at 15° C./180 rpm and then harvested by centrifugation (4° C., 7000 g for 10 minutes). Cell pellets were resuspended in buffer A (200 mM KCl, 1 mM MnCl₂, 50 mM Tris pH 7.5) supplemented with DNase, RNase, lysozyme (Sigma) and Complete EDTA-free protease inhibitor cocktail (Roche). Cells were lysed using a French press at 1500 psi and the lysate incubated at 50° C. for 30 minutes to precipitate host proteins. The lysate was clarified by centrifugation at 125,000 g for 90 minutes. The P. thermopropionicum HmfF was precipitated with 30% saturating ammonium sulphate at 4° C., the supernatant was removed following centrifugation and the pellet solubilized in buffer A and subjected to size exclusion chromatography using a HiPrep S200 column (GE Healthcare) equilibrated with buffer A and 2 ml fractions collected. Samples were subjected to SDS-PAGE analysis and fractions found to contain the purified protein were pooled. Protein was aliquoted and flash frozen until required.

Mutant Reconstitution and Assays.

Reconstitution and Assays were performed anaerobically within a 100% N₂-atmosphere glove box (Belle Technology, UK).

Holo-PtHmfF (e.g., an enzyme with its cofactors) was obtained by reconstituting single expressed HmfF with reduced prFMN under anaerobic conditions as described previously for the UbiD from E. coli in Marshall, S. A., Fisher, K., Ni Cheallaigh, A., White, M. D., Payne, K. A., Parker, D. A., Rigby, S. E., and Leys, D. (2017) Oxidative Maturation and Structural Characterization of Prenylated FMN Binding by UbiD, a Decarboxylase Involved in Bacterial Ubiquinone Biosynthesis. The Journal of biological chemistry 292, 4623-4637. In general, a reaction consisting of 1 mM FMN, 2 mM DMAP (Sigma), 50 μM Fre reductase and 50 μM UbiX in buffer A was started by the addition of 5 mM NADH. Following incubation, the reaction mixture was filtered through 10 k MWCO centrifugal concentrator to remove UbiX and Fre proteins. The filtrate containing the prFMN product was used to reconstitute anaerobic apo-HmfF (e.g., an enzyme without its cofactors) in a 2:1 molar ratio, with excess cofactor being removed using a PD25 desalting column (GE Healthcare). Use of holo-PtHmfF ensures the activity observed in the enzymatic activity assay below is minimally impacted by the presence or absence of cofactors.

Assays were performed by UV-vis spectroscopy using a Cary 50 Bio spectrophotometer (Varian). HmfF was assayed against 1 mM 2,5-furan-dicarboxylic acid (FDCA) or 2,5-pyrrol-dicarboxylic acid (PDCA), in 50 mM KCl, 50 mM NaPi pH6 in a 1 mm path length quartz cuvette at 50° C. The rate of FDCA consumption was monitored at 265 nm and PDCA consumption was monitored at 270 nm.

Primer Table:

| Primer Name | Primer Sequence (5′ to 3′) |
|---|---|
| PtH297N_R | 5′-cagcagcagattttcacggctggccgg-3′ |
| PtH297N_F | 5′-ccggccagccgtgaaaatctgctgctg-3′ |
| PtH297F_R | 5′-gcccagcagcagaaattcacggctggccgg-3′ |
| PtH297F_F | 5′-ccggccagccgtgaatttctgctgctgggc-3′ |
| PtR305Q_R | 5′-agcaggacggcttcctgcgcaataccgccc-3′ |
| PtR305Q_F | 5′-gggcggtattgcgcaggaagccgtcctgct-3′ |

FIG. 7 provides enzymatic activity for the WT and variants in the presence of the FDCA or PDCA substrate in terms of k_{cat}, which is an expression for the amount of cycles completed per enzyme per time unit. PDCA is selected because it is one of the closest structural homologues of FDCA, but contains an NH rather than O in the central aromatic ring. The binding model based on the enzyme structure information predicts the H297 is involved in discriminating for O rather than NH. The assay results for the H297N mutant indicate that it no longer distinguishes between PDCA and FDCA, which supports the model.

In particular, FIG. 7 is a graph of observed k_{cat} values for P. thermopropionicum HmfF wild-type (WT) that has H297 and R305 sites conserved and variants assayed with 1 mM FDCA and 1 mM PDCA (2,5-pyrrol-dicarboxylic acid) as substrates, which demonstrates the decarboxylation activity of the respective enzyme with the respective substrate. As can be seen, WT P. thermopropionicum HmfF is able to discern between FDCA and PDCA, where the decarboxylation activity of FDCA is greater than 4 k_{cat}/s but less than 0.1 k_{cat}/s for PDCA, which is about 2.5% the activity for FDCA. The assays with FDCA and PDCA indicate that the three variants of H297N, H297F, and R305Q are non-functional variant. As can be seen in FIG. 7, the H297F and R305Q variants did not exhibit any measurable decarboxylation activity of either FDCA or PDCA. The mutagenesis at H297 and R305 indicate that substitution of at least one of these two amino acids can generate a non-functional variant that appears to have a negatively impacted binding site for FDCA, which results in substantially diminished decarboxylation reaction activity of FDCA. The diminished decarboxylation reaction activity suggests a corollary negative impact on the binding site of furoic acid and carboxylation reaction to form FDCA.

1. A method for preparing a 2,5-furandicarboxylic acid (“FDCA”) comprising:
a. contacting a HmfF polypeptide comprising an amino acid sequence that has greater than 34% sequence identity with an amino acid sequence set out in SEQ ID NO:1 or SEQ ID NO:2 with furoic acid in the presence of carbon dioxide,
b. wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

2. The method of claim 1, wherein the method is performed in vitro, such as a cell-free lysate system.

3. The method of claim 1, further comprising contacting the HmfF polypeptide in the presence of carbon dioxide in an amount greater than ambient conditions.

4. A vector or plasmid or isolated and purified polynucleotide comprising:
a. a nucleic acid sequence encoding a HmfF polypeptide comprising an amino acid sequence that has greater than 34% sequence identity with an amino acid sequence set out in SEQ ID NO:1 or SEQ ID NO:2,
b. wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2.

5. A recombinant host cell comprising the vector or plasmid or isolated and purified polynucleotide of claim 4.

6. A cell-free lysate composition comprising:
a. a HmfF polypeptide comprising an amino acid sequence that has greater than 34% sequence identity with an amino acid sequence set out in SEQ ID NO:1 or SEQ ID NO:2, wherein the polypeptide has carboxylase and decarboxylase activity and comprises (i) the amino acid corresponding to H297 or a functional substitution thereof, and (ii) at least one of (a) the amino acid corresponding to R305 or a functional substitution thereof; and (b) the amino acid corresponding to R332 or a functional substitution thereof, wherein the position is numbered relative to the amino acid sequence of SEQ ID NO:1 or SEQ ID NO:2;
b. furoic acid; and
c. carbon dioxide.

7. The composition of claim 6 wherein the carbon dioxide is present in an amount of greater than ambient conditions, such as at least 400 ppm.

8. The composition of claim 6, further comprising FDCA.

9. The composition of claim 8 wherein the FDCA is converted from the furoic acid by the HmfF polypeptide.

10. The method of claim 1 wherein the method is performed in a reactor having a pressure of greater than 1 bar.

11. The method of claim 10 wherein the reactor has a temperature suitable for the HmfF polypeptide to catalyze the reaction, such as a temperature in a range of 35-60 degrees C., including 45-55 degrees C.

12. The method of claim 10, wherein the reactor comprises a gas phase comprising carbon dioxide and an aqueous phase comprising furoic acid and the HmfF polypeptide.

13. The method of claim 1, wherein the contacting of the HmfF enzyme and the furoic acid and carbon dioxide takes place at a pressure greater than 1 bar, including in the reactor.

14. The method of claim 1, wherein the functional substitution comprises a conservative substitution.

15. The method of claim 14, wherein the conservative substitution can comprise at least one of (i) the amino acid corresponding to R305K; and (ii) the amino acid corresponding to R332K, wherein the position is numbered relative to the amino acid sequence of SEQ ID NO:1 or SEQ ID NO:2.

16. The method of claim 1, wherein the contact of the HmfF polypeptide with furoic acid and carbon dioxide takes place at a temperature in a range of 37-60 degrees C., including in the reactor.

17. The method of claim 1, wherein the HmfF polypeptide is capable of catalyzing the decarboxylation reaction of FDCA into furoic acid.

18. The method of claim 1, wherein the furoic acid is present in an amount of at least 0.1 mM.

19. The method of claim 1, wherein the reaction conditions comprise a pH in a range of 5-9.

20. The method of claim 1, wherein the carbon dioxide is under supercritical conditions.
